(19) United States
(12) Patent Application Publication
(10) Pub. No.: US 2012/0083460 A1
(43) Pub. Date: Apr. 5, 2012

(54) READILY WATER-SOLUBLE ISOQUERCITRIN COMPOSITION

(75) Inventors: Kazuhiro Emura, Osaka (JP); Hiroshi Oka, Osaka (JP); Hisashi Tanaka, Osaka (JP)

(73) Assignee: SAN EI GEN F.F.I., INC., Toyonaka-shi, Osaka (JP)

(21) Appl. No.: 13/258,537

(22) PCT Filed: Mar. 24, 2010
(86) PCT No.: PCT/JP2010/055105

§ 371 Date: Sep. 22, 2011

(30) Foreign Application Priority Data
Mar. 25, 2009 (JP) 2009-074977

(51) Int. Cl. A61K 31/7048; A61P 39/06

(52) U.S. Cl. 514/27

(57) ABSTRACT

Disclosed is a method for improving the solubility of isoquercitrin in water. Also disclosed is a readily water-soluble isoquercitrin composition which has improved solubility in water due to the method. Specifically disclosed is a method for preparing an isoquercitrin inclusion product, which comprises including isoquercitrin in γ-cyclodextrin in the proportion of 2 to 10 mol of γ-cyclodextrin to 1 mol of isoquercitrin.

### TECHNICAL FIELD

The present invention relates to a readily water-soluble isoquercitrin composition that has improved solubility in water compared to the isoquercitrin itself. The present invention also relates to a method for producing the readily water-soluble isoquercitrin composition. Further, the present invention relates to a method for improving the solubility of isoquercitrin in water, and the absorption of isoquercitrin in the body.

The present invention also relates to various uses of the readily water-soluble isoquercitrin composition, specifically uses as a color deterioration suppressing agent and a flavor deterioration suppressing agent.

### BACKGROUND ART

Flavonol derivatives such as rutin and isoquercitrin generally have an antioxidative effect and radical scavenging effect. Flavonol derivatives are thus used as food additives such as antioxidants, fading preventing agents, and flavor deterioration preventing agents. There are also reports that flavonol derivatives are effective for the prevention of diseases that involve factors such as free radicals and active oxygen.

A problem of rutin and other flavonol derivatives, however, is that these compounds generally have poor solubility in water, and are unstable in aqueous solutions. At high concentration, flavonol derivatives may form precipitates by forming insoluble matter over time in an acidic aqueous solution, or even in an alkaline aqueous solution, in which the flavonol derivatives have high solubility. The poor dissolution stability of flavonol derivatives is detrimental to the appearance of food, and makes the food difficult to be ingested.

Another problem is the low absorption in the body, which makes the efficacy and effect of flavonol derivatives insufficient. Therefore, flavonol derivatives are taken in large amounts to sufficiently obtain their effects.

Accordingly, methods that stably mix poorly water-soluble flavonol derivatives in an aqueous solution at high concentrations have been studied. For example, Patent Literature 1 describes including rutin in β- or γ-cyclodextrin, and thereby improving the solubility of rutin in water. Patent Literature 2 describes including rutin in cyclodextrin under alkaline conditions to improve the rutin solubility in a low temperature range. Patent Literature 3 describes performing an alkali treatment for an isoflavone derivative included in β- or γ-cyclodextrin to further improve solubility in water. Patent Literature 4 describes including a poorly water-soluble flavonoid in β-cyclodextrin in an alkaline aqueous solution or in a mixture of water and organic solvent, or in the presence of a supercritical to subcritical aqueous solvent, and mixing enzyme-treated hesperidin therewith to prepare a water-soluble flavonoid composition that has improved solubility in water.

### CITATION LIST

### Patent Literature

PTL 1: JP-A-59-137499
PTL 2: JP-A-06-054664
PTL 3: JP-A-2004-238336
PTL 4: JP-A-2008-271839

### SUMMARY OF INVENTION

### Technical Problem

As described above, it is known that including poorly water-soluble flavonol derivatives such as rutin in cyclodextrin has certain effects in improving water solubility. However, it is thought that this inclusion method can increase water solubility only by a factor of, at most, about 10 to 15 in the case of rutin; and there is a need for ingenuity to further improve water solubility.

It is an object of the present invention to provide a method for greatly improving the solubility of poorly water-soluble isoquercitrin in water, and a readily water-soluble isoquercitrin composition that has greatly improved water solubility. Another object is to provide various uses of the readily water-soluble isoquercitrin composition.

### Solution to Problem

The present inventors conducted intensive studies to solve the foregoing problems, and found that an isoquercitrin composition obtained by including γ-cyclodextrin in a proportion of 2 to 10 moles per mole of isoquercitrin can have improved water solubility over the non-included isoquercitrin by a factor of 120 or more in terms of isoquercitrin. Further, it was confirmed that the readily water-soluble isoquercitrin composition has better lightfastness (degradation resistance) in acidic drinks than that of the isoquercitrin, and that a drink mixed with the composition does not undergo undesirable changes such as formation of precipitations and turbidity even when preserved under low temperature to 60° C. conditions, regardless of whether the drink is acidic or alkaline. Specifically, it was confirmed that the readily water-soluble isoquercitrin composition can be used to prepare an aqueous food that excels in isoquercitrin preservation stability. The present inventors also confirmed that the readily water-soluble isoquercitrin composition has better absorption in the body than that of the isoquercitrin itself.

The present invention has been completed based on these findings, and has the following aspects.

### (I) Readily Water-Soluble Isoquercitrin Composition

(I-1) A readily water-soluble isoquercitrin composition that includes γ-cyclodextrin in a proportion of 2 to 10 moles per mole of isoquercitrin.
(I-2) A readily water-soluble isoquercitrin composition that includes γ-cyclodextrin in a proportion of 3 to 8 moles per mole of isoquercitrin.
(I-3) The readily water-soluble isoquercitrin composition according to (I-1) or (I-2), wherein the isoquercitrin is an inclusion product of the γ-cyclodextrin.
(I-4) The readily water-soluble isoquercitrin composition according to (I-1) or (I-3), wherein the solubility of the readily water-soluble isoquercitrin composition in water in terms of isoquercitrin under the condition shaking for 40 hours at 25° C., is 120 times or higher, preferably 120 to 220 times higher than the solubility of the isoquercitrin alone.
(I-5) The readily water-soluble isoquercitrin composition according to (I-1) or (I-3), wherein the solubility of the readily water-soluble isoquercitrin composition in water in terms of isoquercitrin under the condition shaking for 40 hours at 25° C., is 12 mg/ml or more, preferably 12 to 25 mg/ml.
(I-6) The readily water-soluble isoquercitrin composition according to (I-2) or (I-3), wherein the solubility of the readily water-soluble isoquercitrin composition in water in terms of isoquercitrin under the condition shaking for 40 hours at 25° C., is 140 times or higher, preferably 140 to 300 times higher than the solubility of the isoquercitrin alone.
(I-7) The readily water-soluble isoquercitrin composition according to (I-2) or (I-3), wherein the solubility of the readily water-soluble isoquercitrin composition in water in terms of isoquercitrin under the condition shaking for 40 hours at 25° C., is 14 mg/ml or more, preferably 14 to 30 mg/ml.
(I-8) The readily water-soluble isoquercitrin composition according to any one of (I-1) to (I-7), wherein the readily water-soluble isoquercitrin composition is a color deterioration suppressing agent.
(I-9) The readily water-soluble isoquercitrin composition according to any one of (I-1) to (I-7), wherein the readily water-soluble isoquercitrin composition is a flavor deterioration suppressing agent.

### (II) Readily Water-Soluble Isoquercitrin Composition Producing Method

(II-1) A method for producing the readily water-soluble isoquercitrin composition of (I-1), (I-3), (I-4) or (I-5), the method comprising including the isoquercitrin in the γ-cyclodextrin in a proportion of 2 to 10 moles of the γ-cyclodextrin per mole of the isoquercitrin.
(II-2) The method according to (II-1), comprising subjecting a mixture that contains 2 to 10 moles of γ-cyclodextrin per mole of isoquercitrin to the following steps:

(1) dissolving the mixture to a heated aqueous solution, and

(2) drying the aqueous solution.
(II-3) The method according to (II-2), comprising the step of clarifying the aqueous solution between the steps (1) and (2).
(II-4) A method for producing the readily water-soluble isoquercitrin composition of (I-2), (I-3), (I-6) or (I-7), the method comprising including the isoquercitrin in the γ-cyclodextrin in a proportion of 3 to 8 moles of the γ-cyclodextrin per mole of the isoquercitrin.

(II-5) The method according to (II-4), comprising subjecting a mixture that contains 3 to 8 moles of γ-cyclodextrin per mole of isoquercitrin to the following steps:

(1) dissolving the mixture in a heated aqueous solution, and

(2) drying the aqueous solution.
(II-6) The method according to (II-5), comprising the step of clarifying the aqueous solution between the steps (1) and (2).
(II-7) The method according to (II-2) or (II-5), wherein the heated aqueous solution has a temperature of 50° C. or more.

### (III) Edible Composition Containing Readily Water-Soluble Isoquercitrin Composition

(III-1) An edible composition that comprises the readily water-soluble isoquercitrin composition of any one of (I-1) to (I-7) in the state of being dissolved in water or in aqueous ethanol.
(III-2) An edible composition according to (III-1), wherein the edible composition is a drink.
(III-3) An edible composition according to (III-1) or (III-2), wherein the edible composition is an acidic drink.
(III-4) An edible composition obtained by solidifying the edible composition of (III-1).

### (IV) Isoquercitrin Water-Solubility Improving Method

(IV-1) A method for improving solubility of isoquercitrin in water, the method comprising isoquercitrin in γ-cyclodextrin in a proportion of 2 to 10 moles of the γ-cyclodextrin per mole of the isoquercitrin, so as to produce an isoquercitrin inclusion product of γ-cyclodextrin.
(IV-2) The method according to (IV-1), comprising subjecting a mixture that contains 2 to 10 moles of γ-cyclodextrin per mole of isoquercitrin to the following steps:

(1) dissolving the mixture in a heated aqueous solution, and

(2) drying the aqueous solution.
(IV-3) The method according to (IV-2), including the step of clarifying the aqueous solution between the steps (1) and (2).
(IV-4) The method according to any one of (IV-1) to (IV-3), wherein the solubility in water at 25° C. in terms of isoquercitrin is improved 120 times or higher, preferably 120 to 300 times from the solubility of the isoquercitrin itself.
(IV-5) A method for improving solubility of isoquercitrin in water, the method comprising including isoquercitrin in γ-cyclodextrin in a proportion of 3 to 8 moles of the γ-cyclodextrin per mole of the isoquercitrin, so as to produce an isoquercitrin inclusion product of γ-cyclodextrin.
(IV-6) The method according to (IV-5), including comprising subjecting a mixture that contains 3 to 8 moles of γ-cyclodextrin per mole of isoquercitrin to the following steps:

(1) dissolving the mixture in a heated aqueous solution, and

(2) drying the aqueous solution.
(IV-7) The method according to (IV-6), comprising the step of clarifying the aqueous solution between the steps (1) and (2).
(IV-8) The method according to any one of (IV-5) to (IV-7), wherein the solubility in water at 25° C. in terms of isoquercitrin is improved 140 times or higher, preferably 140 to 300 times from the solubility of the isoquercitrin itself.

### (V) Color Deterioration Suppressing Agent and Color Deterioration Suppressing Method

(V-1) A color deterioration suppressing agent for a colorant, that contains the readily water-soluble isoquercitrin composition of any one of (I-1) to (I-7) as an active ingredient.
(V-2) A color deterioration suppressing agent according to (V-1), wherein the colorant suppressed from fading is a natural colorant.
(V-3) A color deterioration suppressing agent according to (V-1) or (V-2), wherein the colorant suppressed from fading is an anthocyanin-based colorant, a flavonoid-based colorant, a carotenoid-based colorant, a quinone-based colorant, an azaphilone-based colorant, or gardenia blue colorant.
(V-4) A color deterioration suppressing agent according to any one of (V-1) to (V-3), wherein the color deterioration suppressing agent is against photoirradiation.
(V-5) A colorant preparation that comprises a colorant with a color deterioration suppressing agent of any one of (V-1) to (V-4).
(V-6) A colorant preparation according to (V-5), wherein the color is a natural colorant.
(V-7) A colorant preparation according to (V-6), wherein the color is an anthocyanin-based colorant, a flavonoid-based colorant, a carotenoid-based color, a quinone-based colorant, an azaphilone-based colorant, or gardenia blue colorant.
(V-8) A fading-suppressed colored food or drink that contains a color deterioration suppressing agent of any one of (V-1) to (V-4).
(V-9) A color deterioration suppressing method for a colorant or a colorant-containing composition, the method comprising having the colorant or the colorant-containing composition coexist with the readily water-soluble isoquercitrin composition of any one of (I-1) to (I-7).
(V-10) A color deterioration suppressing method according to (V-9), wherein the colorant is an anthocyanin-based colorant, a flavonoid-based colorant, a carotenoid-based colorant, a quinone-based colorant, an azaphilone-based colorant, or gardenia blue colorant.

### (VI) Flavor Deterioration Suppressing Agent and Flavor Deterioration Suppressing Method

(VI-1) A flavor deterioration suppressing agent that includes the readily water-soluble isoquercitrin composition of any one of (I-1) to (I-7) as an active ingredient.
(VI-2) A flavor deterioration suppressing agent according to (VI-1), wherein the flavor is a citrus- or milk-based flavor.
(VI-3) A scented product that contains a flavor component with a flavor deterioration suppressing agent of (VI-1) or (VI-2).
(VI-4) A scented product according to (VI-3), wherein the flavor component has a citrus- or milk-based flavor.
(VI-5) A scented product according to (VI-3) or (VI-4), wherein the scented product is a food or a drink.
(VI-6) A flavor deterioration suppressing method for a flavor component-containing composition subject to flavor deterioration, the method comprising having the readily water-soluble isoquercitrin composition any one of (I-1) to (I-7) is coexisted with said composition.
(VI-7) The flavor deterioration suppressing method according to (VI-6), wherein the flavor component has a citrus- or milk-based flavor.

### (VII) Method for Improving Isoquercitrin Absorption in the Body

(VII-1) A method for improving the body's absorption of isoquercitrin taken orally, the method comprising including isoquercitrin in γ-cyclodextrin in a proportion of 2 to 10 moles of the γ-cyclodextrin per mole of the isoquercitrin.
(VII-2) The method according to (VII-1), comprising subjecting a mixture that contains 2 to 10 moles of γ-cyclodextrin per mole of isoquercitrin to the following steps:

(1) dissolving the mixture in a heated aqueous solution, and

(2) drying the aqueous solution.
(VII-3) The method according to (VII-2), comprising the step of clarifying the aqueous solution between the steps (1) and (2).
(VII-4) The method according to anyone of (VII-1) to (VII-3), wherein the isoquercitrin is included in the γ-cyclodextrin so as to prepare a readily water-soluble isoquercitrin composition whose solubility in water at 25° C. in terms of isoquercitrin is improved 120 times or higher, preferably 120 to 300 times from the solubility of the isoquercitrin itself.
(VII-5) A method for improving the body's absorption of isoquercitrin taken orally, comprising including isoquercitrin in γ-cyclodextrin in a proportion of 3 to 8 moles of the γ-cyclodextrin per mole of the isoquercitrin.
(VII-6) The method according to (VII-5), comprising subjecting a mixture than contains 3 to 8 moles of γ-cyclodextrin per mole of isoquercitrin to the following steps:

(1) dissolving the mixture in a heated aqueous solution, and

(2) drying the aqueous solution.
(VII-7) The method according to (VII-6), comprising the step of clarifying the aqueous solution between the steps (1) and (2).
(VII-8) The method according to any one of (VII-5) to (VII-7), wherein the isoquercitrin is included in the γ-cyclodextrin so as to prepare a readily water-soluble isoquercitrin composition whose solubility in water at 25° C. in terms of isoquercitrin is improved 140 times or higher, preferably 140 to 300 times from the solubility of the isoquercitrin itself.

### Advantageous Effects of Invention

The isoquercitrin composition of the present invention including γ-cyclodextrin in a proportion of 2 to 10 moles, preferably 3 to 8 moles per mole of isoquercitrin has improved solubility in water over the non-included isoquercitrin by a factor of 120 or more, preferably 140 or more. Further, the readily water-soluble isoquercitrin composition has better lightfastness (degradation resistance) in acidic drinks than that of the isoquercitrin. Further, a drink mixed with the composition does not undergo undesirable changes such as formation of precipitations and turbidity even when preserved under low temperature to 60° C. conditions, regardless of whether the drink is acidic or alkaline. It is therefore possible to prepare an aqueous food that excels in preservation stability. Furthermore, the readily water-soluble isoquercitrin composition is superior to the isoquercitrin itself with regard to fading suppressing effect, flavor deterioration suppressing effect and absorption in the body.

### BRIEF DESCRIPTION OF DRAWINGS

FIG. 1 represents the results of Experiment Example 1 in which the solubility of isoquercitrin in water was examined using the isoquercitrin itself (non-included isoquercitrin), and various isoquercitrin compositions that contained varying amounts of γ-cyclodextrin with respect to isoquercitrin; the upper diagram represents the isoquercitrin solubility (mg/ml) obtained from using the isoquercitrin and isoquercitrin composition, the lower diagram represents the isoquercitrin solubility (mg/ml) of the isoquercitrin composition as the relative ratio (fold) with respect to the reference solubility (mg/ml) 1 of the isoquercitrin itself (isoquercitrin:γ-CD=1:0).

FIG. 2 represents the results of Experiment Example 2 which calculated the relative ratio (fold) of the water solubility of various flavonoid compositions prepared by varying the mixed amount of γ-cyclodextrin for various flavonoids (isoquercitrin, quercetin, myricetin, rutin, and naringin) with respect to the reference solubility (mg/ml) 1 of the flavonoid itself (flavonoid:γ-CD=1:0).

FIG. 3 represents the results of Experiment Example 4 which measured the concentrations (μg/ml) of the total metabolites (quercetin, isorhamnetin, tamarixetin) in blood plasma after 1, 2, and 3 hours from the oral administration of the isoquercitrin itself (open square) and the isoquercitrin inclusion product of γ-cyclodextrin (solid square).

### DESCRIPTION OF EMBODIMENTS

### (1) Readily Water-Soluble Isoquercitrin Composition, and Method of Production Thereof

The isoquercitrin of the present invention is a flavonol glycoside that has a glucose attached to position 3 of quercetin, as represented by the formula below. In rutin, glucosyl rhamnose is attached to position 3 of quercetin.

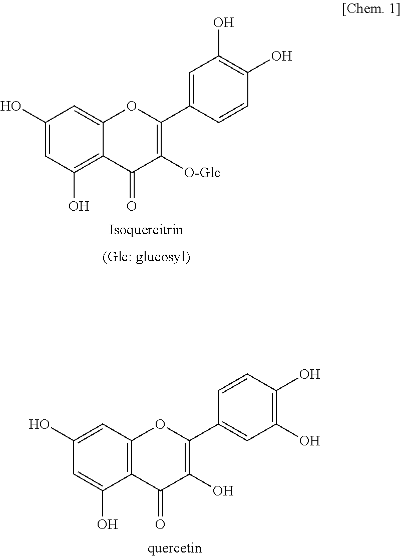

The isoquercitrin is commercially available from, for example, Tokyo Chemical Industry Co., Ltd., Funakoshi Corporation, and Sigma-Aldrich Japan.

Cyclodextrins are crown-shaped nonreducing maltooligosaccharides with 6 to 12 glucose molecules linked together in a ring with α-1,4 glucoside bonds. Cyclodextrins are produced from starch acted upon by cyclodextrin producing enzymes that originate in, for example, Bacillus macerans. Typical cyclodextrins include α-cyclodextrin with six glucose molecules, β-cyclodextrin with seven glucose molecules, and γ-cyclodextrin with eight glucose molecules. The cyclodextrin suited for the preparation of the isoquercitrin composition of the present invention is γ-cyclodextrin, which can be used to desirably obtain the effects of the present invention. Branched or methylated cyclodextrins with improved solubility, or mixtures of α-cyclodextrin, β-cyclodextrin, and γ-cyclodextrin also can be used.

The isoquercitrin composition of the present invention can easily be prepared by mixing isoquercitrin and γ-cyclodextrin. The mixing method may be, for example, a kneading method, a dissolving method, or a mixing and pulverizing method. The preferred method is a dissolving method.

### (a) Kneading Method

γ-Cyclodextrin is mixed with isoquercitrin in a proportion of 2 to 10 moles per mole of isoquercitrin. Then, water is added in 0.5 to 5 times the amount of the mixture. The product is then kneaded into a paste, and dried.

Kneading may be performed typically at 5 to 100° C.; however, a more efficient treatment is possible at 100 to 160° C. with the use of a pressurized vessel. Kneading time is not particularly limited, and may be about 30 minutes to 3 hours. Devices such as a mixing-grinding machine, a bowl mill and an emulsification equipment may be used for kneading. The paste after the inclusion may be dried and formed into a powder, as required, using methods such as reduced-pressure drying, and drum drying.

### (b) Dissolving Method

γ-Cyclodextrin is mixed with isoquercitrin in a proportion of 2 to 10 moles per mole of isoquercitrin. The mixture is heat dissolved in water in about 1 to 50 mass, and the resulting aqueous solution is dried.

When use in transparent drinks such as soft drinks, it is preferable that the heat dissolving of the isoquercitrin and γ-cyclodextrin in water is followed by a clarification treatment such as filtration, before drying the resulting aqueous solution.

The mixture may be dissolved in water typically at 50 to 100° C., preferably 70 to 100° C. Further, to help ease the process, the dissolving may be performed at 100° C. and higher, preferably 100 to 165° C., more preferably 110 to 140° C., by an indirect heat treatment using, for example, a plate heater, a pressurized heat treatment using, for example, retort sterilization, or a direct heat treatment using steam such as in steam injection, and steam infusion.

Further, an aqueous solution of lower alcohols such as methanol, ethanol, and isopropanol (25 mass % or less) may be used instead of water.

The method used to dry the aqueous solution is not particularly limited. The aqueous solution is typically dried using methods such as spray drying, reduced-pressure drying, drum drying, and freeze drying.

The isoquercitrin and γ-cyclodextrin may be mixed under alkaline conditions to adjust the amount of the isoquercitrin included. Typically, the mixture may be placed under alkaline conditions by using methods that use one or more alkalis used as food additives, such as sodium hydroxide, sodium carbonate, sodium bicarbonate, sodium acetate, sodium citrate, potassium hydroxide, potassium carbonate, potassium phosphate, calcium hydroxide, calcium carbonate, and kansui(brine). The preferred alkaline condition is pH 7 to 10, preferably pH 7 to 8.

Inclusion under alkaline conditions is not limited, and may be performed as follows. One to three parts by mass of γ-cyclodextrin is dispersed in 5 to 10 parts by mass of water, and completely dissolved by stirring the mixture under the applied heat of 60 to 80° C. A predetermined amount of isoquercitrin is then added to the solution. At the maintained temperature of 60 to 80° C., the solution is gently stirred to make the alkali typically about 0.5 to 5 mass %, and further stirred for 0.1 to 2 hours at the adjusted pH of 7 to 10. This is followed by addition of acids such as sulfuric acid and citric acid to adjust the pH to 4 to 6. As a result, an isoquercitrin inclusion product of γ-cyclodextrin (isoquercitrin composition) is obtained. The isoquercitrin composition prepared this way may be directly used as the solution, or may be dried and formed into a powder using methods such as freeze drying, spray drying, reduced-pressure drying, and drum drying.

### (c) Mixing and Pulverizing Method

γ-Cyclodextrin is added to isoquercitrin a proportion of 2 to 10 moles per mole of the isoquercitrin. These are mixed in a solid state by being crushed with a device such as a high-speed pulverizer.

The isoquercitrin and rcyclodextrin used for the production of the inclusion product (isoquercitrin composition) may be mixed in a proportion of typically 2 to 10 moles of γ-cyclodextrin per mole of isoquercitrin, regardless of the mixing method used. The preferred proportion is 3 to 8 moles, more preferably 4 to 7 moles, further preferably 5 moles.

The isoquercitrin inclusion product of γ-cyclodextrin (isoquercitrin composition) obtained in the methods (a) to (c) is characterized by the greatly improved (increased) solubility in water compared to the isoquercitrin itself. The solubility (in terms of isoquercitrin) of the isoquercitrin composition in water is 120 times or higher, preferably 140 times or higher, more preferably 170 times or higher, further preferably 200 times or higher than the solubility of the isoquercitrin itself in water under the condition for 40 hours at. The upper limit is not particularly limited, and is about 220 times based on the Experiment Examples below.

Accordingly, the “readily water-soluble isoquercitrin composition” as used herein is an isoquercitrin composition whose solubility in water as measured in terms of isoquercitrin by being added to water and shaken at 25° C. for 40 hours is 120 times or higher, preferably 140 times or higher, more preferably 170 times or higher, further preferably 200 times or higher than the solubility of the isoquercitrin itself (non-inclusion product) measured under the same conditions. The upper limit is not particularly limited, and is about 220 times based on the Experiment Examples below.

In terms of the absolute amount of isoquercitrin, the “readily water-soluble isoquercitrin composition” is an isoquercitrin composition whose solubility in water as measured in terms of isoquercitrin by being shaken and dissolved at 25° C. for 40 hours is 12 mg/ml or more, preferably 14 mg/ml or more, more preferably 17 mg/ml or more, further preferably 20 mg/ml or more, particularly preferably 23 mg/ml or more. The upper limit is not particularly limited, and is about 25 mg/ml based on the Examples below.

With the improved solubility in water, the readily water-soluble isoquercitrin composition of the present invention can be stably dissolved in large amounts in a water-soluble edible composition (described later), and an edible composition dissolving high concentrations of isoquercitrin can be prepared. The stable dissolution is the characteristic effect obtained with the use of the readily water-soluble isoquercitrin composition of the present invention, as will be described in Experiment Example 2. The readily water-soluble isoquercitrin composition of the present invention can thus be stably dissolved without causing the isoquercitrin to deposit, regardless of the pH (acidic to alkaline) or the temperature (for example, low temperature to 60° C.) of the edible composition. Further, with the readily water-soluble isoquercitrin composition of the present invention, degradation of the isoquercitrin can be significantly suppressed even when preserved by being dissolved in, for example, an acidic water-soluble composition. The readily water-soluble isoquercitrin composition of the present invention can thus be used to prepare a water-soluble edible composition that excels in isoquercitrin preservation stability.

Further, because of the high solubility in water, the readily water-soluble isoquercitrin composition has advantages over isoquercitrin compositions mixed without being included. Specifically, the readily water-soluble isoquercitrin composition melts more easily in the mouth even when taken orally in the form of a solid, a granule, or a powder; and thus causes less grittiness in the mouth, and is easier to ingest.

Further, as will be described in Experiment Example 6, the readily water-soluble isoquercitrin composition of the present invention has greatly improved absorption in the body, making it possible to effectively utilize the physiological effect (antioxidative effect) of the isoquercitrin in the body.

### (2) Edible Composition Containing Readily Water-Soluble Isoquercitrin Composition

As described above, the isoquercitrin composition of the present invention has improved solubility in water, and can thus be stably mixed in large amounts in a water-soluble composition, making it possible to prepare an edible composition dissolving high concentrations of isoquercitrin.

The edible composition of the present invention contains liquid or semiliquid edible compositions compatible with water and dissolving the readily water-soluble isoquercitrin composition of the present invention. Preferably, the edible composition of the present invention is a composition that dissolves isoquercitrin in a proportion of 0.01 mass % or more (0.1 mg/ml), preferably 0.011 mass % or more (0.11 mg/ml) under the condition at 25° C. The upper limit solubility of the isoquercitrin itself in water under acidic condition at 25° C. is 0.0102 mass % (0.102 mg/ml), as will be described in Experiment Example 1.

The liquid or semiliquid edible composition of the present invention is a composition that is obtained by dissolving the readily water-soluble isoquercitrin composition of the present invention in water or aqueous alcohol without causing the composition to deposit. The isoquercitrin content is not particularly limited, so long as this is satisfied. For example, the upper limit solubility in water under the acidic condition at 25° C. maybe 2.2 mass % (22 mg/ml), based on the Experiment Examples below.

Here, “under the condition at 25° C.” as used herein is not intended to limit the temperature of the composition of interest, but is rather a reference temperature used to evaluate the solubility of the composition of the present invention.

The liquid or semiliquid edible composition of the present invention may contain a solvent that is 100% water, or may be an aqueous alcohol (preferably, aqueous ethanol) that contains no greater than 25 mass % of alcohols such as ethanol as the solvent.

The edible composition of the present invention contains edible compositions of a solid form obtained by solidifying the liquid or semiliquid edible composition. The solidification process is not particularly limited, and may be performed by using ordinary solidifying means, such as cooling, freezing, heating, and drying (including freeze drying, and spray drying). The edible composition of the present invention may be any edible composition solidified in this manner.

Examples of the edible composition of the present invention include oral medicine (drink, syrup, etc.), quasi drug (mouth wash, etc.), health food (drink, tablet, etc.), food with health claims (e.g., food with nutrient function claims, food for specified health uses, etc.), and food and drink. Examples of food and drink include, but are not limited to, milk beverages, lactobacillus beverages, fruit juice-containing soft drinks, soft drinks, carbonated beverages, fruit juice beverages, vegetable drinks, vegetable•fruit drinks, alcoholic beverages, powdered beverages, concentrated drinks for dilution with water, coffee drinks, shiruko (sweet red-bean soup with pieces of rice cake) beverage, black tea beverages, green tea beverages, barley tea beverages, oolong tea beverages, hatomugi (adlay) tea beverages, soba (buckwheatk) tea beverages, puer tea beverages, and other such beverages; custard pudding, milk pudding, souffle pudding, fruit juice-containing pudding, and other such puddings; jellies, Bavarian cream, yogurt, and other such desserts; ice cream, ice milk, lacto-ice, milk ice cream, fruit juice-containing ice cream, soft serve ice cream, ice lollipops, sherbet, and other such frozen concoctions; chewing gum, bubble gum, and other such gums (stick gum and sugar-coated gum granules); marble chocolate and other such coated chocolates, as well as strawberry chocolate, blueberry chocolate, melon chocolate, and other flavored chocolates, and other such chocolates; Ramune (tablet candies); hard candy (including bonbons, butterballs, and marbles), soft candy (including caramel, nougat, gummy candy, and marshmallow), drops, taffy, and other such candies; hard biscuits, cookies, okaki (cracker made from glutinous rice), senbei (cracker made from regular rice), and other such baked snacks (hereinafter, “snacks”); miso soup, sumashi jiru (a clear soup), consomme soup, potage soup, and other such soups; asazuke (lightly-pickled vegetables), soy sauce pickles, salt pickles, miso pickles, kasuzuke (fish or vegetables pickled in sake lees), kojizuke (rice malt pickles), nukazuke (vegetables pickled in brine and fermented rice bran), vinegar pickles, mustard pickles, moromizuke (unrefined miso pickles), pickled plum, fukujinzuke (sliced vegetables pickled in liquid preparation containing soy sauce and dyed red), shibazuke (assorted vegetables hashed and pickled in salt), pickled ginger, plum vinegar pickles, and other such pickles; vinaigrette dressings, non-oil dressings, ketchup, gravy, sauce, and other such sauces; strawberry jam, blueberry jam, marmalade, apple jam, apricot jam, preserves, and other such jams; red wine and other such fruit wines; candied cherries, apricots, apples, strawberries, peaches, and other such processed fruits; ham, sausage, roast pork, and other such processed meats; fish meat ham, fish meat sausage, ground fish meat, boiled fish paste, chikuwa (tubular fish cakes), hanpen (a cake of pounded fish), satsumaage (fried fish cakes), datemaki (rolled omelets mixed fish paste), whale bacon, and other ground marine products; konjac, tofu, and other such processed farm products; butter, margarine, cheese, whip cream, and other such dairy-fatty products; udon noodles, hiyamugi (cold wheat noodles), somen (thin wheat noodles), soba, Chinese soba noodles, spaghetti, macaroni, rice noodles, harusame (thin noodles made from bean starch), wonton, and other such pastas; as well as various types of side dishes and processed foods such as dried bread-like pieces of wheat gluten and denbu (mashed and seasoned fish).

The readily water-soluble isoquercitrin composition of the present invention has greatly improved solubility in water, and can thus provide effects particularly when prepared by being added to an aqueous transparent edible composition. Preferably, such liquid or semiliquid edible compositions are, for example, products that require transparency, such as drinks, jelly-like food, jams, fruit sauces, or beverages. Preferred examples of solid-form edible compositions include products that require transparency, such as hard candies and jelly food, and products dissolved in water or warm water before consumed, such as powdered beverages, solid soups, and powdered soups. The liquid property (pH) is not particularly limited, and is, for example, 2 to 7, more preferably 2.5 to 6.5.

Another advantage of the readily water-soluble isoquercitrin composition of the present invention owning to the greatly improved solubility in water is that it does not deposit even when added to an aqueous edible composition in high concentration. Examples of edible compositions suited in this regard include coating chocolates, syrups for sugar coating (sugar solution), candies, frozen concoctions, chocolates, and gummy candies. Other examples include tofu, konjac, seaweeds (these are liquids or semiliquids during manufacture, but are solidified into solid-form edible compositions by, for example, cooling, freezing, heating, or drying).

Adding the isoquercitrin composition of the present invention to the edible composition does not require a dedicated step. For example, the isoquercitrin composition may be appropriately added with the raw material in the early stages of the food and drink manufacturing steps, or may be added in the middle or later stages of the manufacturing steps. The addition method is not particularly limited either, and may be selected from ordinary methods such as kneading, dissolving, dipping, dispersing, spraying, and coating, according to the type and properties of the edible compositions.

### (3) Water Solubility Improving Method for Isoquercitrin

The present invention also provides a method for improving the solubility of isoquercitrin in water. The method can be realized by including isoquercitrin in γ-cyclodextrin in a proportion of 2 to 10 moles of γ-cyclodextrin per mole of isoquercitrin, as described above. The proportion of γ-cyclodextrin per mole of isoquercitrin is preferably 3 to 8 moles, more preferably 4 to 7 moles, further preferably 5 moles.

The inclusion of the isoquercitrin in the γ-cyclodextrin may be performed in the same manner as in the readily water-soluble isoquercitrin composition producing method described in section (1) above.

The isoquercitrin composition obtained this way has greatly improved solubility in water compared to the non-included isoquercitrin. The solubility of the isoquercitrin composition in water in terms of isoquercitrin is improved 120 times or more, preferably 140 times or more, more preferably 170 times or more, further preferably 200 times or more from the solubility of the isoquercitrin itself in water under conditions shaking for 40 hours at 25° C. The upper limit is not particularly limited, and is about 220 times based on the Examples below.

The method for improving the water solubility of the isoquercitrin of the present invention also can be used for preventing the deposition of the remaining isoquercitrin in the α-glucosyl isoquercitrin prepared from the isoquercitrin by the action of glycosyltransferase such as cyclodextrin glucanotransferase.

### (4) Color Deterioration Suppressing Agent and Color Deterioration Suppressing Method

### (4-1) Color Deterioration Suppressing Agent

The color deterioration suppressing agent of the present invention contains the readily water-soluble isoquercitrin composition of the present invention as the active ingredient.

The color deterioration suppressing agent of the present invention may contain the readily water-soluble isoquercitrin composition as aforementioned. The color deterioration suppressing agent of the present invention may contain the readily water-soluble isoquercitrin composition as the sole component, or may also contain other components such as diluents, carriers, and other such additives.

The diluents and carriers are not particularly limited, as long as they do not interfere with the effects of the present invention. Examples include sugars such as sucrose, glucose, dextrin, starches, trehalose, lactose, maltose, corn syrup, and liquid sugar; alcohols such as ethanol, propylene glycol, and glycerine; sugar alcohols such as sorbitol, mannitol, xylitol, erythritol, and maltitol; polysaccharides such as gum arabic, gum ghatti, xanthan gum, carrageenan, guar gum, gellan gum, and celluloses; and water. Examples of additives include chelating agents and other auxiliary agents, flavorings, spice extracts, antiseptics, preservatives, pH adjusters, stabilizers, and oxidation inhibitors.

A wide range of compounds used as food additives can be used as the oxidation inhibitors used as additives. Examples include, but are not limited to, ascorbic acids such as L-ascorbic acid and sodium L-ascorbate; ascorbic acid esters such as L-ascorbyl stearate, and L-ascorbyl palmitate; erythorbic acids such as erythorbic acid and salts thereof (e.g., sodium erythorbate); sulfites such as sodium sulfite, sodium hyposulfite, sodium pyrosulfite, and potassium pyrosulfite; tocopherols such as α-tocopherol and mix tocopherol; dibutylhydroxytoluene (BHT), butylhydroxyanisole (BHA); ethylenediaminetetraacetic acids such as calcium disodium ethylenediaminetetraacetate, and disodium ethylenediaminetetraacetate; gallic acids such as gallic acid and propyl gallate; citric acids such as citric acid and isopropyl citrate; sulfur dioxide; and various plant extracts such as hollyhock flower extract, Aspergillus terreus extract, licorice oil extract, clove extract, essential oil-removed fennel extract, horseradish extract, sage extract, dropwort extract, tea extract, tempeh extract, coffee bean extract, sunflower seed extract, pimento extract, grape seed extract, blueberry leaf extract, propolis extract, Hego(Cyathea fauriei)-Ginkgo(Ginkgo biloba) leaf extract, pepper extract, garden balsam extract, eucalyptus leaf extract, gentian root extract, enzymatically decomposed apple extract, sesame oil extract, rapeseed oil extract, rice bran oil extract, enzymatically decomposed rice bran, Chinese bayberry extract, rutin extract (red bean whole plant, Styphnolobium japonicum, soba whole plant extract), and rosemary extract. Other examples include γ-oryzanol, ellagic acid, guaiac gum, sesamolin, sesamol, Melaleuca oil, amino acid-sugar reaction product, chlorogenic acid, phytic acid, ferulic acid, tocotrienol, rapeseed oil extract, dokudami (Houttuynia cordata) extract, sesame oil unsaponifiable, hesperetin, catechin, morin, enzyme-treated rutin, quercetin, and enzyme-treated isoquercitrin.

For convenience in use, it is preferable that the color deterioration suppressing agent prepared with the diluent, carrier, or additive contain the readily water-soluble isoquercitrin composition (as a dried product) in at least 0.01 mass %, preferably 0.1 to 20 mass %, more preferably 1 to 15 mass % in terms of the isoquercitrin amount.

The form of the color deterioration suppressing agent of the present invention is not particularly limited, and the color deterioration suppressing agent may be prepared in any form, including, for example, a solid form such as a powder, a granule, and a tablet; a solution form such as a liquid and an emulsion; and a semi-solid form such as a paste.

The colorant of the color deterioration suppressing agent of the present invention includes a wide range of colors, both synthetic and natural. Synthetic colors may be synthetic colorants, including tar colors such as Food Red No. 2, Food Red No. 3, Food Red No. 40, Food Red No. 102, Food Red No. 104, Food Red No. 105, Food Red No. 106, Food Yellow No. 4, Food Yellow No. 5, Food Blue No. 1, Food Blue No. 2, and Food Green No. 3; inorganic colorants such as ferric oxide and titanium dioxide; natural colorant derivatives such as sodium norbixinate, potassium norbixinate, copper chlorophyll, sodium copper chlorophyllin, and potassium copper chlorophyllin; and synthetic natural colors such as β-carotene, riboflavin, riboflavin butyrate ester, and 5′-riboflavin phosphate ester sodium.

Examples of natural colorants include carotenoid-based colorants such as annatto colorant, gardenia yellow colorant, Dunaliella carotene, carrot carotene, palm oil carotene, marigold colorant, tomato colorant, and paprika colorant; anthocyanin-based colorants such as red cabbage colorant, red radish colorant, perilla colorant, hibiscus colorant, grape juice colorant, grape skin colorant, purple sweet potato colorant, purple corn colorant, elderberry colorant, and boysenberry; flavonoid-based colors such as cacao color, kaoliang colorant, rosewood colorant, onion colorant, tamarind colorant, persimmon colorant, carob colorant, sweetroot colorant, sappanwood colorant, safflower red colorant, and safflower yellow colorant; quinone-based colorants such as madder colorant, cochineal colorant, shikon colorant (root extract from Lithospermum erythrorhizon), and lac colorant; porphyrin-based colorants such as chlorophyllin, chlorophyll, and spirulina colorant; diketone-based colorants such as turmeric colorant; betacyanin-based colors such as beet red; and azaphilone-based colorants such as Monascus colorant. Other examples include, Monascus yellow colorant, caramel, gardenia blue colorant, Gardenia red colorant, gold, silver, and aluminum-based colorants. Preferably, the color deterioration suppressing agent of the present invention may be used for natural colorants. More preferably, the color deterioration suppressing agent has a wide range of applications in products containing the natural colorants exemplified above, specifically, natural colorants such as carotenoid-based colorants, anthocyanin-based colorants, flavonoid-based colorants, quinone-based colorants, azaphilone-based colorants, and gardenia blue. The color deterioration suppressing agent is thus useful in suppressing or preventing the fading of these colorants.

The specific products (colored products) to which the color deterioration suppressing agent of the present invention are applicable are not particularly limited, as long as the products contain the colorants above. Examples include colorant preparations, food and drink (food products), cosmetics, drugs, quasi drugs, and feeds, preferably colorant preparations and food and drink (food products). Use of the color deterioration suppressing agent of the invention for such products (colored products) is described in the following section (4-2).

### (4-2) Colored Products Containing the Color Deterioration Suppressing Agent

The present invention provides a colored product that uses the readily water-soluble isoquercitrin composition of the present invention as the color deterioration suppressing agent. By containing the composition, the colored product can have the effect to significantly suppress the color fading phenomenon of colorant, specifically the fading phenomenon resulting from exposure to light.

As used herein, the term “colored” refers to not only colorants imparted to a product by intentionally adding a colorant, but a broad range of colorants that originate in the colorants naturally present in the material of food and drink products, for example, such as in fruit juice and vegetable juice. Further, the “colored product” as used herein encompasses a variety of products imparted with colors, particularly with the natural colorants exemplified above. Specific examples include colorant preparations, colorant-containing colored food and drink, colorant-containing colored cosmetics, colorant-containing colored drugs, colorant-containing colored quasi drugs, and colorant-containing colored feeds.

The colorant preparation of the present invention may be one that contains one or more of the synthetic or natural colorants above in addition to the readily water-soluble isoquercitrin composition of the present invention, preferably, colorant preparations containing one or more natural colorants. The preferred colorant preparation is one that contains at least one natural colorant selected from the group of various colorants that belong to carotenoid-based colorants, anthocyanin-based colorants, flavonoid-based colorants, quinone-based colorants, azaphilone-based colorants, and gardenia blue colorant.

The proportion of the readily water-soluble isoquercitrin composition mixed in the colorant preparation is not particularly limited, as long as the effects of the present invention are obtained. For example, the isoquercitrin composition may be contained in the colorant preparation in a proportion of 0.01 mass % or more, preferably 0.1 to 20 mass %, more preferably 1 to 15 mass % in terms of the isoquercitrin amount.

The colorant preparation of the present invention includes at least a color and the readily water-soluble isoquercitrin composition, and may additionally include an oxidation inhibitor, a chelating agent, a flavoring or a spice extract, an antiseptic, a preservative, a pH adjuster, or a stabilizer, as required.

The colorant preparation of the present invention may be produced by methods commonly used for the preparation of various color preparations, except for mixing the readily water-soluble isoquercitrin composition in any of the production steps. The method for mixing the readily water-soluble isoquercitrin composition, or the order in which the components are mixed are not particularly limited. However, considering that the colorant is under the influence of heat and light to various extents, it is preferable that the composition be mixed with other materials at early stages of the colorant preparation production steps, preferably before the heat-treatment step or before exposure to light.

The food and drink of the present invention are not particularly limited, as long as a color is imparted, preferably based on the natural colorant. For example, the various foods and drinks exemplified in section (2) Edible Composition Containing Readily Water-Soluble Isoquercitrin Composition may be used. The preferred food and drink are drinks and jellies.

The food and drink of the present invention can be produced according to methods commonly used for the production of various foods and drinks, except for mixing the readily water-soluble isoquercitrin composition in any of the production steps. The method for mixing the readily water-soluble isoquercitrin composition, or the order in which the components are mixed are not particularly limited. However, considering that the colorant is under the influence of heat and light to various extents, it is preferable that the readily water-soluble isoquercitrin composition be mixed at early stages of the production steps, preferably before the heat-treatment step or before exposure to light.

The amount of the color deterioration suppressing agent of the present invention added to various colored products such as food and drink, cosmetics, drugs, quasi drugs, and feeds is not particularly limited, provided that the color fading phenomenon can be prevented. Various amounts can be appropriately selected and decided taking into consideration such factors as the type and the content of the colorant contained in the colored product, the type and the intended use of the product, and the type of the components contained in the product. For example, when the colored product is adjusted in such a manner that the colorant to be suppressed from fading has an absorbance of 0.05 to 1 (color valency (E^{10%}_{1 cm})=0.005 to 0.1) at the maximum absorption wavelength, the color deterioration suppressing agent is preferably mixed with the colored product in 0.001 mass % or more, preferably 0.001 to 0.1 mass %, more preferably 0.002 to 0.05 mass % in terms of the isoquercitrin amount.

The color valency means the concentration of the colorant in a colored material (colored solution), and is generally represented by the absorbance value of a 10 w/v % solution (E^{10%}_{1 cm}) converted from the measured absorbance of the colored material (colored solution) in the visible range at the maximum absorption wavelength. Specifically, the color valency (E^{10%}_{1 cm}) can be obtained by first adjusting the concentration of the measured colored material (colored solution) to confine the absorbance within a 0.3 to 0.7 range, and then measuring the absorbance at the maximum absorption wavelength using a cell with a layer length of 1 cm. The measured absorbance is then converted to an absorbance for a 10 w/v % concentration of the colored material (colored solution). (see the Specifications and Standards for Food Additives, 8th ed., 17. Color Valency Measurement Method).

### (4-3) Fading Suppressing Method

The present invention also provides a method for suppressing the fading of a colorant or various compositions containing a colorant.

The colorant of the present invention is any of the synthetic and natural colorants above, preferably any of the natural colorants above, more preferably any of the carotenoid-based colorants, anthocyanin-based colorants, flavonoid-based colorants, quinone-based colorants, azaphilone-based colorants, and gardenia blue. Specifically, the fading suppressing method of the present invention excels in suppressing the fading phenomenon caused by irradiation of the colorant with light (excels in lightfastness), as will be described later in Experiment Examples.

As used herein, various compositions containing a colorant (colorant-containing compositions) means a wide range of compositions that contain a colorant, preferably a natural colorant. Specific examples are various colored products, such as the colorant preparations, food and drink, cosmetics, drugs, quasi drugs, and feeds exemplified above.

The present invention can be effected by having the colorant or the colorant-containing composition coexist with the readily water-soluble isoquercitrin composition or the color deterioration suppressing agent of the present invention. The coexisting form of these components is not particularly limited, as long as these are present in contact with each other. For example, such a coexisting form may be obtained by mixing the colorant or the colorant-containing composition with the readily water-soluble isoquercitrin composition that has the fading suppressing effect. For example, when the colorant-containing composition is a colorant preparation or a food or drink, the coexisting state can be achieved by mixing the readily water-soluble isoquercitrin composition as one of the material components during the production of the colorant preparation or the food or drink. This is also possible in other colored products such as cosmetics, drugs, quasi drugs, and feeds.

The proportion of the readily water-soluble isoquercitrin composition used for the colorant or the colorant-containing composition is not particularly limited as long as the effects of the present invention can be obtained, and may be appropriately adjusted according to the type of the color of interest. The proportion of the readily water-soluble isoquercitrin composition used for the colorant-containing composition is not particularly limited, and may be 0.001 mass %, preferably 0.001 to 0.1 mass %, more preferably 0.002 to 0.05 mass % in terms of the isoquercitrin amount, when the colorant-containing composition is adjusted in such a manner that the colorant to be suppressed from fading has an absorbance of 0.05 to 1 (color valency (E^{10%}_{1 cm})=0.005 to 0.1) at the maximum absorption wavelength.

The fading suppressing method of the present invention can significantly suppress the fading of the colorant or the colorant-containing composition. The fading suppressing method of the present invention particularly excels in suppressing fading that occurs as a result of the photoirradiation of the carotenoid-based colorant, anthocyanin-based colorant, flavonoid-based colorant, quinone-based colorant, azaphilone-based colorant, and gardenia blue colorant, or of the compositions containing these colorants, and can render the colorant or the colorant-containing composition photofading-resistant (lightfast).

As used herein, “photofading-resistant” refers to the property that resists fading even under the influence of sunlight or artificial light (such as fluorescent light). Specifically, the term “photofading-resistant” refers to the property to significantly suppress the fading of the colorant or the colorant-containing composition placed under light (sunlight, fluorescent light, etc.) present in normal storage conditions, compared to colorants or colorant-containing compositions that do not contain the color deterioration suppressing agent. Examples of such conditions include exposing the colorant or the colorant-containing composition to sunlight for 5 minutes to several hours, or to fluorescent light for 1 day to 6 months.

### (5) Flavor Deterioration Suppressing Agent and Flavor Deterioration Suppressing Method

### (5-1) Flavor Deterioration Suppressing Agent

The flavor deterioration suppressing agent of the present invention contains the readily water-soluble isoquercitrin composition of the present invention as the active ingredient.

The flavor deterioration suppressing agent of the present invention may contain the readily water-soluble isoquercitrin composition as the sole component, or may also contain other components such as diluents, carriers, and other such additives, provided that the isoquercitrin composition is contained.

The diluents and carriers are not particularly limited, as long as they do not interfere with the effects of the present invention. Examples include sugars such as sucrose, glucose, dextrin, starches, trehalose, lactose, maltose, corn syrup, and liquid sugar; alcohols such as ethanol, propylene glycol, and glycerine; sugar alcohols such as sorbitol, mannitol, xylitol, erythritol, and maltitol; polysaccharides such as gum arabic, gum ghatti, xanthan gum, carrageenan, guar gum, gellan gum, and celluloses; and water. Examples of additives include oxidation inhibitors, chelating agents and other auxiliary agents, flavorings, spice extracts, antiseptics, preservatives, pH adjusters, and stabilizers.

A wide range of compounds used as food additives can be used as the oxidation inhibitors used as additives. Examples include, but are not limited to, ascorbic acids such as L-ascorbic acid and sodium L-ascorbate; ascorbic acid esters such as L-ascorbyl stearate, and L-ascorbyl palmitate; erythorbic acids such as erythorbic acid and salts thereof (e.g., sodium erythorbate); sulfites such as sodium sulfite, sodium hyposulfite, sodium pyrosulfite, and potassium pyrosulfite; tocopherols such as α-tocopherol and mix tocopherol; dibutylhydroxytoluene (BHT), butylhydroxyanisole (BHA); ethylenediaminetetraacetic acids such as calcium disodium ethylenediaminetetraacetate, and disodium ethylenediaminetetraacetate; gallic acids such as gallic acid and propyl gallate; citric acids such as citric acid and isopropyl citrate; sulfur dioxide; and various plant extracts such as hollyhock flower extract, Aspergillus terreus extract, licorice oil extract, clove extract, essential oil-removed fennel extract, horseradish extract, sage extract, dropwort extract, tea extract, tempeh extract, coffee bean extract, sunflower seed extract, pimento extract, grape seed extract, blueberry leaf extract, propolis extract, Hego(Cyathea fauriei)-Ginkgo(Ginkgo biloba) leaf extract, pepper extract, garden balsam extract, eucalyptus leaf extract, gentian root extract, enzymatically decomposed apple extract, sesame oil extract, rapeseed oil extract, rice bran oil extract, enzymatically decomposed rice bran, Chinese bayberry extract, rutin extract (red bean whole plant, Styphnolobium japonicum, soba whole plant extract), and rosemary extract. Other examples include γ-oryzanol, ellagic acid, guaiac gum, sesamolin, sesamol, Melaleuca oil, amino acid-sugar reaction product, chlorogenic acid, phytic acid, ferulic acid, tocotrienol, rapeseed oil extract, dokudami (Houttuynia cordata) extract, sesame oil unsaponifiable, hesperetin, catechin, morin, enzyme-treated rutin, quercetin, and enzyme-treated isoquercitrin.

For convenience in use, it is preferable that the flavor deterioration suppressing agent prepared with the diluent, carrier, or additive contain the readily water-soluble isoquercitrin composition in 0.01 mass % or more, preferably 0.1 to 20 mass %, preferably 1 to 15 mass % in terms of the isoquercitrin amount.

The form of the flavor deterioration suppressing agent of the present invention is not particularly limited, and the flavor deterioration suppressing agent may be prepared in any form, comprising, for example, a solid form such as a powder, a granule, and a tablet; a solution form such as a liquid and an emulsion; and a semi-solid form such as a paste.

The flavor component of the flavor deterioration suppressing agent of the present invention encompasses flavor components that form flavorings, which may be natural flavorings (plant natural flavorings, animal natural flavorings), or synthetic flavorings.

Specific examples of the flavor components include those forming citrus-based flavorings such as orange, lemon, and grapefruit; fruit-based flavorings such as apple, grape, peach, banana, and pineapple; milk-based flavorings such as milk, butter, cheese, and yogurt; vanilla-based flavorings; tea-based flavorings such as black tea and green tea; coffee-based flavorings; mint-based flavorings; spice-based flavorings such as herb, pepper, and wasabi; nut-based flavorings; meat-based flavorings such as beef, pork, and chicken; marine products flavorings such as fish and shellfish, and crustaceans; Western liquor-based flavorings such as wine, whisky, and brandy; flower-based flavorings such as rose, lavender, and jasmine; vegetable-based flavorings such as onion, garlic, and cabbage; flavorings for dishes such as meat dish, seafood, and vegetable cuisine; and other such flavorings. The preferred flavor component is one that forms citrus-based flavorings and milk-based flavorings. In general, flavorings cannot be reproduced from a single scent component, and multiple scent components are used. For example, a flavoring is prepared by blending a variety of scent components with the main component source substances that characterize different types of scent. The source substances for different types of scent are known, and can be prepared by a person with ordinary skill in the art (see, for example, the reference Kaori no Sougou Jiten, Japan Flavor & Fragrance Materials Association, ed., Asakura Publishing Co., Ltd., Dec. 10, 1998).

As will be described in Experiment Examples, the scent deterioration suppressing agent of the present invention has been shown to effectively suppress the flavor deterioration phenomenon caused by light or heat in food products, comprising food and drink containing citrus-based flavorings, and food and drink containing milk-based flavorings (lightfastness and heat resistance). Thus, the flavor deterioration suppressing agent of the present invention can be used in a wide range of products (scented products) that contain various flavor components, preferably flavor components that form citrus-based flavorings or milk-based flavorings. The flavor deterioration suppressing agent of the present invention is therefore useful in suppressing or preventing flavor deterioration in these products.

The flavor deterioration suppressing agent of the present invention can be used in a wide range of products (flavor-containing products, scented products, flavored products; hereinafter, collectively referred to as “scented products”) for the suppression of flavor deterioration, particularly the flavor deterioration caused by light or heat. Examples of such scented products include flavorings, food and drink, cosmetics, drugs, quasi drugs, and feeds, preferably flavorings, food and drink, and cosmetics. The preferred form is an aqueous form, specifically solutions such as drinks, lotions, and liquid formulation, particularly aqueous solutions.

The flavor deterioration suppressing agent of the present invention can prevent flavor deterioration by being added and mixed with products that have the flavors imparted by flavor components such as flavorings, or with products that naturally contain flavor components. Use of the flavor deterioration suppressing agent of the invention for such scented products will be described in sections (5-2) and (5-3) below.

### (5-2) Scented Products Containing Flavor Deterioration Suppressing Agent

The present invention provides a scented product that contains the readily water-soluble isoquercitrin composition as the flavor deterioration suppressing agent. By containing the readily water-soluble isoquercitrin composition, the scented product has the effect of significantly suppressing flavor deterioration phenomena, particularly the flavor deterioration phenomenon caused when exposed to light or heat.

As used herein, the term “scented” refers not only to scent imparted by intentionally adding a flavor component (flavoring) to the product, but a broad range of flavors that originate in the flavor components naturally present in the material of food and drink products, such as in, for example, fruit juice and vegetable juice. Further, the “scented product” as used herein encompasses a variety of products scented with the flavor components, particularly with the flavorings exemplified above. Specific examples include flavorings themselves, flavoring preparations, food and drink, cosmetics, drugs, quasi drugs, and feeds.

Examples of preferred products include flavoring; food and drink claimed to have a commercial value in giving a flavor sensation in the mouth; cosmetics such as lipsticks and lip balms; oral pharmaceutical preparations; quasi drugs such as tooth pastes, mouth washes, and mouth odor preventive agents. Flavorings and food and drink are more preferred.

The flavoring of the present invention may be any flavoring, including natural flavorings (plant natural flavorings, animal natural flavorings) and synthetic flavorings, both in simple and preparation forms, regardless of the producing method and form (water-soluble flavorings, oily flavorings, emulsion flavorings, powder flavorings), and whether it is used for food or cosmetics.

Preferably, the flavoring is a citrus-based flavoring such as orange, lemon, and grapefruit flavorings, or a milk-based flavoring such as butter, cheese, and yogurt flavorings.

The flavoring may be categorized according to the intended use, as follows. Beverage flavorings used for carbonated beverages, fruit drinks, tea/coffee-based drinks, milk beverage, Lactobacillus beverages, beverages with function claims, and other such beverages; sweets flavorings used for frozen concoctions, candies or desserts, chewing gums, baked snacks, and other such sweets; flavorings for dairy or fatty products such as yogurt, butter or margarine, and cheese; soup flavorings; flavor enhancing flavorings used for products such as miso, soy sauce, sauce, gravy, and dressing; flavorings for processed meat; flavorings for processed fishery products; flavorings for cooked food; food flavorings such as frozen food flavorings; cigarette flavorings; flavorings for mouth products; drug flavorings; feed flavorings; and flavorings for industrial use.

The proportion of the flavor deterioration suppressing agent mixed in the flavoring is not particularly limited, as long as the effects of the present invention can be obtained. For flavorings generally used in 0.05 to 0.2 mass % for the flavored product, the preferred proportion of the readily water-soluble isoquercitrin composition for the flavoring is 0.01 mass % or more, preferably 0.1 to 20 mass %, more preferably 1 to 15 mass % in terms of the isoquercitrin amount. Although the upper limit is not limited in terms of the effect of the present invention, the proportion for liquid flavorings is, for example, preferably 10 mass % or less, because the excess addition may be detrimental to the natural flavor of the flavored product, or may cause the insoluble matter to deposit.

The flavoring obtained in this manner can be provided as a flavoring that does not undergo flavor deterioration during the production steps or distribution or in long storage, and that is resistant to light, heat, or other factors that promote deterioration. Further, the flavoring can not only impart a desired flavor to various products such as food and drink, cosmetics, drugs, quasi drugs, and feeds, but significantly prevent flavor deterioration caused by factors such as heat, light, and oxygen, particularly flavor deterioration caused by heat, in products such as food and drink, cosmetics, drugs, quasi drugs, and feeds.

The flavoring of the present invention can be produced according to methods commonly used for flavorings, except for mixing the readily water-soluble isoquercitrin composition in any of the production steps. The method for mixing the readily water-soluble isoquercitrin composition, or the order in which the components are mixed are not particularly limited. However, considering that the flavoring is under the influence of heat and light to various extents, it is preferable that the readily water-soluble isoquercitrin composition be mixed with various materials in the early stages of the flavoring production steps, preferably before the heat-treatment step or before exposure to light.

The food and drink of the present invention are not particularly limited, as long as they are scented, preferably by containing the flavoring (flavor component). More preferably, the food and drink are those having citrus-based or milk-based scent. Examples of the food and drink include milk beverages, Lactobacillus beverages, fruit juice-containing soft drinks, soft drinks, carbonated beverages, fruit juice beverages, vegetable drinks, vegetable/fruit drinks, alcoholic beverage, powdered beverages, concentrated drinks for dilution with water, coffee drinks, shiruko (sweet red-bean soup with pieces of rice cake) beverage, black tea beverages, green tea beverages, barley tea beverages, oolong tea beverages, hatomugi (adlay) tea beverages, soba (buckwheatk) tea beverages, Dattan soba (tartary buckwheat) tea beverages, puer tea beverages, and other such beverages; custard pudding, milk pudding, souffle pudding, fruit juice-containing pudding, and other such puddings; jellies, Bavarian cream, yogurt, and other such desserts; ice cream, ice milk, lacto-ice, milk ice cream, fruit juice-containing ice cream, soft serve ice cream, ice lollpops, sherbet, and other such frozen concoctions; chewing gum, bubble gum, and other such gums (stick gum and sugar-coated gum granules); marble chocolate and other such coated chocolates, as well as strawberry chocolate, blueberry chocolate, melon chocolate, and other flavored chocolates, and other such chocolates; hard candy (including bonbons, butterballs, and marbles), soft candy (including caramel, nougat, gummy candy, and marshmallow), drops, taffy, and other such candies; hard biscuits, cookies, okaki (cracker made from glutinous rice), senbei (cracker made from regular rice), and other such baked snacks (hereinafter, “snacks”); miso soup, sumashi jiru (a clear soup), consomme soup, potage soup, and other such soups; asazuke (lightly-pickled vegetables), soy sauce pickles, salt pickles, miso pickles, kasuzuke (fish or vegetables pickled in sake lees), kojizuke (rice malt pickles), nukazuke (vegetables pickled in brine and fermented rice bran), vinegar pickles, mustard pickles, moromizuke (unrefined miso pickles), pickled plum, fukujinzuke (sliced vegetables pickled in liquid preparation containing soy sauce and dyed red), shibazuke (assorted vegetables hashed and pickled in salt), pickled ginger, plum vinegar pickles, and other such pickles; vinaigrette dressings, non-oil dressings, ketchup, gravy, sauce, and other such sauces; strawberry jam, blueberry jam, marmalade, apple jam, apricot jam, preserves, and other such jams; red wine and other such fruit wines; candied cherries, apricots, apples, strawberries, peaches, and other such processed fruits; ham, sausage, roast pork, and other such processed meats; fish meat ham, fish meat sausage, ground fish meat, boiled fish paste, chikuwa (tubular fish cakes), hanpen (a cake of pounded fish), satsumaage (fried fish cakes), datemaki (rolled omelets mixed fish paste), whale bacon, and other ground marine products; butter, margarine, cheese, whip cream, and other such dairy•fatty products; udon noodles, hiyamugi (cold wheat noodles), somen (thin wheat noodles), soba, Chinese soba noodles, spaghetti, macaroni, rice noodles, harusame (thin noodles made from bean starch), wonton, and other such pastas; as well as various types of side dishes and processed foods such as dried bread-like pieces of wheat gluten and denbu (mashed and seasoned fish). Preferably, the food and drink are beverages and sweets.

The food and drink of the present invention can be produced according to methods commonly used for the production of food and drink, except for mixing the readily water-soluble isoquercitrin composition in any of the production steps. The method for mixing the readily water-soluble isoquercitrin composition, or the order in which the components are mixed are not particularly limited. However, it is preferable that the readily water-soluble isoquercitrin composition be mixed with various materials at early stages of the production steps, preferably before the heat-treatment step or before exposure to light.

Examples of the cosmetics of the present invention include cosmetics that contain flavorings, particularly the flavorings (flavor components) above, including skin cosmetics (such as lotions, emulsions, and creams), lipsticks, sunscreen cosmetics, and makeup cosmetics. Examples of the drugs include drugs that contain flavorings, particularly the flavorings (flavor components) above, including tablets, capsule formulations, drinkable preparations, troches, and gargling solutions. Examples of the quasi drugs comprise quasi drugs that contain flavorings, particularly the flavorings (flavor components) above, including tooth pastes, mouth washes, and mouth odor preventive agents. Examples of the feeds include feeds that contain flavorings, particularly the flavorings (flavor components) above, including various pet foods such as cat food and dog food, and feeds for aquarium fish and farmed fish. These are non-limiting examples.

The cosmetics, drugs, quasi drugs, feeds, and other such products can be produced according to methods commonly used for these products, except for mixing the readily water-soluble isoquercitrin composition in any of the production steps. The time to mix the readily water-soluble isoquercitrin composition mixed with the cosmetics, drugs, quasi drugs, or feeds is not particularly limited. However, it is preferable that the readily water-soluble isoquercitrin composition be mixed with various materials at early stages of the flavoring production steps, preferably before the heat-treatment step or before exposure to light.

The amount of the flavor deterioration suppressing agent of the present invention added to various scented products such as food and drink, cosmetics, drugs, quasi drugs, and feeds is not particularly limited, as long as the deterioration of the flavor component contained in these products can be prevented. The type and the content of the flavor component contained in the scented product can be appropriately selected and decided taking into consideration factors such as the type and the intended use of the product, and the components contained therein. For example, the flavor deterioration suppressing agent (readily water-soluble isoquercitrin composition) may be mixed so that the readily water-soluble isoquercitrin composition is contained in the scented product in a proportion of 0.001 mass % or more, preferably 0.001 to 0.lmass %, more preferably 0.002 to 0.05 mass % in terms of the isoquercitrin amount.

### (5-3) Flavor Deterioration Suppressing Method

The present invention also provides a flavor deterioration suppressing method for various compositions that contain a flavoring or a flavor component.

The flavoring of the present invention may be any flavoring, including natural flavorings (plant natural flavorings, animal natural flavorings) and synthetic flavorings, both in simple and preparation forms, regardless of the producing method and form (water-soluble flavorings, oily flavorings, emulsion flavorings, powder flavorings), and whether it is used for food or cosmetics.

Preferably, the flavoring is a citrus-based flavoring such as orange, lemon, and grapefruit flavorings, or a milk-based flavoring such as butter, cheese, and yogurt flavorings.

As used herein, various compositions that contain a flavoring (flavoring-containing compositions, scented compositions) contain a wide range of compositions that contain the flavoring, preferably the citrus- or milk-based flavor component. Specific examples include various scented products such as the flavorings, food and drink, cosmetics, drugs, quasi drugs, and feeds above.

The method of the present invention can be effected by having the scented product coexist with the readily water-soluble isoquercitrin composition or the flavor deterioration suppressing agent of the present invention. The coexisting form of these components is not particularly limited, as long as these are present in contact with each other. For example, such a coexisting form may be obtained by mixing the readily water-soluble isoquercitrin composition or the flavor deterioration suppressing agent of the present invention with the scented product. When the scented product is a flavoring or a food or drink, the coexisting state can be achieved by mixing the readily water-soluble isoquercitrin composition or the flavor deterioration suppressing agent of the present invention as one of the material components during the production of the flavoring or the food or drink. This is also possible in other scented products such as cosmetics, drugs, quasi drugs, and feeds.

The proportion of the readily water-soluble isoquercitrin composition or the flavor deterioration suppressing agent of the present invention used for the scented product is not particularly limited as long as the effects of the present invention can be obtained, and may be appropriately adjusted according to the type of the flavoring of interest. The proportion of the readily water-soluble isoquercitrin composition or the flavor deterioration suppressing agent of the present invention used for the scented product is not particularly limited, and may be such that the readily water-soluble isoquercitrin composition is contained in the scented product in a proportion of 0.001 mass % or more, preferably 0.001 to 0.1 mass %, more preferably 0.002 to 0.05 mass % in terms of the isoquercitrin amount.

The flavor deterioration suppressing method of the present invention can significantly suppress the flavor deterioration of the scented product.

The flavor deterioration suppressing method of the present invention excels in the effect of suppressing the flavor deterioration caused by light or heat in flavor component-containing compositions, particularly citrus-based flavor component-containing compositions, and can impart heat resistance or light resistance to compositions that contain such flavorings.

As used herein, “heat resistance” refers to the property that resists flavor deterioration (including reduction and alteration) even under the influence of heat. Specifically, the term “heat resistance” refers to the property to significantly suppress the flavor deterioration of the flavoring or flavoring-containing composition placed under the heat (increased temperature and heat) present in the normal storage conditions or in production steps, compared to flavorings or flavoring-containing compositions that do not contain the flavor deterioration suppressing agent. Examples of such conditions include exposing the flavoring or the flavoring-containing composition to a temperature of 60° C. for several tens of hours to 1 month, or 40° C. for 1 day to 6 months.

As used herein, “light resistance” refers to the property that resists flavor deterioration (including reduction and alteration) even under the influence of sunlight or artificial light (such as fluorescent light). Specifically, the term “light resistance” refers to the property to significantly suppress the flavor deterioration of the flavoring or flavoring-containing composition placed under the light (such as sunlight and fluorescent light) present in the normal storage conditions, compared to flavorings or flavoring-containing compositions that do not contain the flavor deterioration suppressing agent. Examples of such conditions include exposing the flavoring or the flavoring-containing composition to sunlight for 5 minutes to several hours, or to fluorescent light for 1 day to 6 months.

(6) Method for Improving isoquercitrin Absorption in the Body

The present invention also provides a method for improving the body's absorption of the isoquercitrin taken orally. The method can be achieved by including isoquercitrin in γ-cyclodextrin in a proportion of 2 to 10 moles of γ-cyclodextrin per mole of isoquercitrin to form an isoquercitrin inclusion. The proportion of γ-cyclodextrin per mole of isoquercitrin is preferably 3 to 8 moles, more preferably 4 to 7 moles, further preferably 5 moles.

The inclusion of the isoquercitrin in the γ-cyclodextrin may be performed in the same manner as in the readily water-soluble isoquercitrin composition producing method described in section (1) above.

The isoquercitrin composition of the present invention may contain the isoquercitrin composition as the sole component, or may also contain other components such as a diluent, a carrier, and other such additives, provided that the isoquercitrin composition is contained.

The diluent or carrier is not particularly limited, as long as it does not interfere with the effects of the present invention. Examples include sugars such as sucrose, glucose, dextrin, starches, trehalose, lactose, maltose, corn syrup, and liquid sugar; alcohols such as ethanol, propylene glycol, and glycerine; sugar alcohols such as sorbitol, mannitol, xylitol, erythritol, and maltitol; polysaccharides such as gum arabic, gum ghatti, xanthan gum, carrageenan, guar gum, gellan gum, and cellulose; and water. The additives may be auxiliary agents such as chelating agents.

For convenience in use, it is preferable that the isoquercitrin composition prepared with the diluent, carrier, or additive contain the readily water-soluble isoquercitrin composition (as a dried product) in 0.01 to 50 mass %, preferably 0.1 to 30 mass % in terms of the isoquercitrin amount.

The form of the isoquercitrin composition is not particularly limited, and may be prepared in any form, including, for example, a solid form such as a powder, a granule, and a tablet; a solution form such as a liquid and an emulsion; and a semi-solid form such as a paste.

The isoquercitrin composition obtained as above has greatly improved metabolic absorption in the body over that of the non-included isoquercitrin. For example, as will be described in Experiment Example 3, the isoquercitrin composition of the present invention can improve the isoquercitrin absorption in the body as high as about 4 times the absorption of the isoquercitrin taken alone, as measured after 2 hours from the administration.

When used as a food with function claims, the isoquercitrin composition may be used in 20 mg to 40 g/day/60 kg B.W., preferably 40 mg to 4 g/day/60 kg B.W., more preferably 50 mg to 1 g/day/60 kg B.W. in terms of the isoquercitrin.

### EXAMPLES

The following specifically describes the substance of the present invention using the Experiment Examples and Examples below. It should be noted that the descriptions below merely represent one aspect of the present invention, and do not limit the present invention in any way. In the following, the symbol “%” means “mass %”, unless otherwise stated. Further, the symbols “IQC”, “γ-CD”, and “IQC-CD” mean isoquercitrin, γ-cyclodextrin, and the isoquercitrin inclusion product of cyclodextrin, respectively. Further, the products with the symbol “*” are San-Ei Gen F.F.I., Inc. products, and the product names with the symbol “**” are the registered trademarks of San-Ei Gen F.F.I., Inc.

### Preparation Example 1

### Preparation of IQC

A flower bud (250 g) of the Fabaceae plant Styphnolobium japonicum was immersed in hot water (2,500 ml, 95° C. or higher) for 2 hours, and the filtrate after filtration was obtained as a first extract. The filtered residue was further immersed in hot water to obtain a second extract. The first and second extracts were mixed, and cooled to 30° C. or below. The precipitated component was filtered out, and water washed, recrystallized, and dried to obtain rutin (22.8 g) of 95% or higher purity.

The rutin (20 g) was dispersed in water (400 ml), and the pH was adjusted to 4.9 with a pH adjuster. Then, 0.12 g of naringinase (naringinase Amano, 3,000 U/g; Amano Enzyme Inc.) was added to the dispersion to perform a reaction. After maintaining the mixture at 72° C. for 24 hours, the reaction solution was cooled to 20° C., and the resulting precipitate was filtered. The precipitate (solid content) was water washed and dried, and IQC (13.4 g) was collected.

### Example 1

### Preparation of Readily Water-Soluble IQC Composition

IQC (the IQC obtained in Preparation Example 1 was used in this Example and the rest of the Examples below) and γ-CD (produced by Wacker Chemical; used in this Example and the rest of Examples below) were mixed at a mole ratio of 1:5 (a total mass of 15 g). After adding 200 ml of tap water, the mixture was heated to about 90° C., and stirred for 15 min to dissolve the solid component. The solution was filtered through a filter paper, and dried to concentrate using an evaporator. The resulting dry solid was powdered with a mixer to prepare a powdery IQC composition (14 g).

### Experiment Example 1

### Evaluation of IQC Composition Solubility in Water

The IQC and γ-CD were used in the proportions (mole ratios) presented in Table 1, and twenty different powdery IQC compositions (Samples 1 to 20) were prepared according to the method of Example 1.

The powdery IQC composition (Samples 1 to 20) or a control IQC (non-inclusion) was added to water (20 ml) in a 100-ml Erlenmeyer flask while stirring. The Samples were added until they could not be dissolved further and formed deposits. Each solution was shaken at 25° C. for 40 hours, and centrifuged at 9,000 rpm for 10 min to collect the supernatant. The supernatant was appropriately diluted with a 0.1% phosphoric acid aqueous solution, and absorbance (340 nm) was measured. From the measured absorbance, the IQC concentration (mg/ml) in the supernatant was calculated as the solubility of the IQC composition (Samples 1 to 22) and IQC (non-inclusion), using a standard curve created beforehand in the manner described below. Further, the IQC solubility of the IQC composition (Samples 1 to 20) relative to the reference solubility (mg/ml) 1 of the IQC (non-inclusion) was also calculated (hereinafter, “solubility (fold)”).

### Standard Curve Creating Method (Spectrophotometer)

1) IQC (50 mg) was accurately weighed, and dissolved in methanol to make the volume precisely 100 ml.
2) The solution was appropriately diluted with a 0.1% phosphoric acid aqueous solution to prepare IQC solutions of 0.0001, 0.0005, 0.001, 0.005, and 0.01 mg/ml concentrations.
3) The absorbance of the standard solution was measured with a spectrophotometer.
4) A standard curve was created based on the IQC content in the IQC solution, and the measured absorbance value.

The results are presented in Table 1 and in FIG. 1 (solubility (mg/ml) and solubility (fold)).

TABLE 1

| | IQC:γ-CD = 1:X | | | Solubility | |
|---|---|---|---|---|---|
| | Mole | Mass | | (in terms of | Solubility |
| Sample | ratio (x) | ratio (x) | pH | isoquercitrin, mg/ml) | (fold) |
| Control | 0 | 0 | 4.74 | 0.10 | 1 |
| Sample 1 | 1 | 2.8 | 4.76 | 7.45 | 73.0 |
| Sample 2 | 2 | 5.6 | 4.90 | 12.30 | 120.6 |
| Sample 3 | 3 | 8.4 | 4.82 | 14.55 | 142.7 |
| Sample 4 | 4 | 11.2 | 4.73 | 17.48 | 171.4 |
| Sample 5 | 5 | 14.0 | 5.20 | 21.72 | 213.0 |
| Sample 6 | 6 | 16.8 | 5.15 | 17.86 | 175.2 |
| Sample 7 | 7 | 19.6 | 5.16 | 17.19 | 168.5 |
| Sample 8 | 8 | 22.4 | 5.18 | 15.53 | 152.3 |
| Sample 9 | 9 | 25.2 | 5.21 | 12.80 | 125.5 |
| Sample 10 | 10 | 28.0 | 5.38 | 13.04 | 127.9 |
| Sample 11 | 11 | 30.8 | 5.35 | 11.27 | 110.5 |
| Sample 12 | 12 | 33.6 | 5.33 | 10.54 | 103.4 |
| Sample 13 | 13 | 36.4 | 5.48 | 9.86 | 96.6 |
| Sample 14 | 14 | 39.2 | 5.50 | 11.24 | 110.2 |
| Sample 15 | 15 | 42.0 | 5.66 | 11.04 | 108.3 |
| Sample 16 | 16 | 44.8 | 5.39 | 10.28 | 100.8 |
| Sample 17 | 17 | 47.6 | 5.64 | 8.30 | 81.4 |
| Sample 18 | 18 | 50.4 | 5.65 | 8.47 | 83.1 |
| Sample 19 | 19 | 53.2 | 5.71 | 6.95 | 68.1 |
| Sample 20 | 20 | 56.0 | 5.74 | 7.25 | 71.1 |

It was found from these results that the use of 2 to 10 moles of γ-CD per mole of IQC greatly improves the IQC solubility by a factor of 120 or more (about 12 mg/ml or more) compared to dissolving the IQC itself (0.1020 mg/ml solubility). It was also found that the use of 3 to 8 moles, preferably 4 to 7 moles, more preferably about 5 moles of γ-CD per mole of IQC greatly improves the IQC solubility by a factor of 140 or more (about 14 mg/ml or more), 170 or more (about 17 mg/ml or more), and 200 or more (about 20 mg/ml or more), respectively, compared to using the IQC itself.

### Reference Experiment Example

### Evaluation of Water Solubility with α- and β-Cyclodextrins

Instead of γ-CD, α-cyclodextrin (α-CD) and β-cyclodextrin (β-CD) (both from Nihon Shokuhin Kako Co., Ltd.) were used to prepare IQC inclusions, and the solubility of the compositions in water was evaluated.

Specifically, an IQC composition containing 5 moles of α-CD (reference sample 1), and an IQC composition containing 5 moles of β-CD (reference sample 2) per mole of IQC were prepared according to the method of Example 1, and the solubility of each composition in water (25° C.) was determined as an IQC solubility (mg/ml) according to the method of Experiment Example 1. The solubility (mg/ml) of the IQC itself was also determined as a control, and the solubility (fold) was calculated from the relative ratio of the two.

The results are presented in Table 2 along with the result of sample 5 evaluated in Experiment Example 1.

TABLE 2

| | | Solubility (in terms of | Solubility |
|---|---|---|---|
| Sample | | isoquercitrin, mg/ml) | (fold) |
| Control | IQC alone | 0.10 | 1 |
| Reference | IQC:α-CD = 1:5 | 0.26 | 2.7 |
| sample 1 | (mole ratio) | | |
| Reference | IQC:β-CD = 1:5 | 1.12 | 11.7 |
| sample 2 | (mole ratio) | | |
| Sample 5 | IQC:γ-CD = 1:5 | 21.72 | 213.0 |
| (Experiment | (mole ratio) | | |
| Example 1) | | | |

As can be seen from the results, the IQC solubility in water is much lower in the compositions using α-CD and β-CD (reference Samples 1 and 2) than in sample 5 using γ-CD, confirming that the improved water solubility of the IQC composition of the present invention is specific to the use of γ-CD.

### Experiment Example 2

### Evaluation of the Solubility of Other Flavonoids in Water

γ-CD and the various flavonoids (IQC, quercetin, myricetin, rutin, and naringin) presented in Table 3 were used in the proportions (mole ratios) given in the table to prepare powder compositions according to the method of Example 1.

The solubility (mg/ml) of each composition in water in terms of the flavonoid was then determined according to the method of Experiment Example 1. The solubility (mg/ml) of the flavonoid itself in water was also determined as a control, and the solubility (fold) was calculated from the relative ratio of the two.

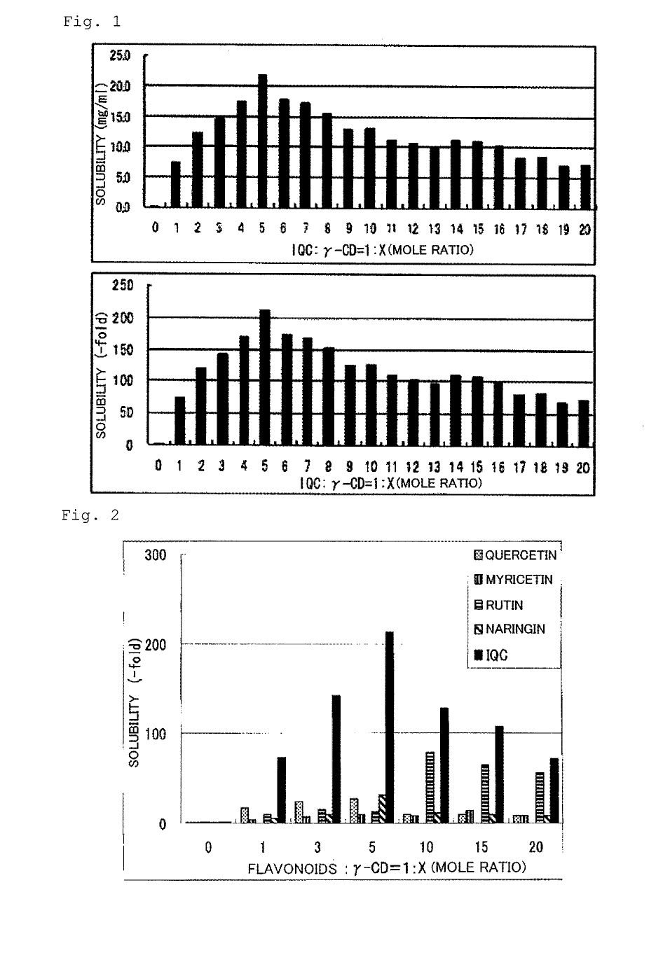

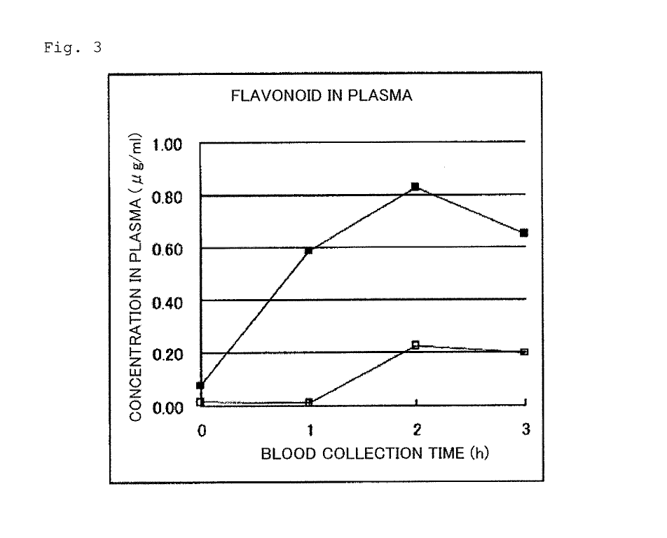

The results (solubility (fold)) are presented in Table 3 and FIG. 2.

TABLE 3

| Flavonoid:γ-CD = 1:X | Solubility (fold) (in terms of flavonoids) | | | | |
|---|---|---|---|---|---|
| (mole ratio) | IQC | Quercetin | Myricetin | Rutin | Naringin |
| 0 | 1 | 1 | 1 | 1 | 1 |
| 1 | 73.0 | 17.2 | 5.0 | 10.7 | 5.4 |
| 3 | 142.7 | 24.9 | 7.6 | 16.1 | 10.7 |
| 5 | 213.0 | 27.7 | 10.6 | 13.5 | 31.2 |
| 10 | 127.9 | 10.4 | 9.2 | 78.3 | 10.8 |
| 15 | 108.3 | 9.9 | 14.0 | 64.3 | 10.6 |
| 20 | 71.1 | 8.4 | 9.2 | 56.2 | 8.7 |

As can be seen from the results, the solubility in water is much lower in Samples using the flavonoids, namely, quercetin, myricetin, rutin, and naringin than in using IQC, confirming that the improved water solubility of the composition of the present invention is specific to the combined use of γ-CD and IQC.

### Experiment Example 3

### Evaluation of Preservation Stability (Presence or Absence of Deposits)

IQC and γ-CD were used in the 1:5 proportion (mole ratio) to prepare a powder composition (sample 1) according to the method of Example 1. The composition was dissolved in acid and sugar solutions of the composition below (pH values of 3, 6, and 9) in a final IQC concentration of 0.1% or 0.05%. Each solution was hot-packed in a 200-ml PET bottle (93° C.). The drink so prepared was allowed to cool, and placed under 50° C., room temperature (25±2° C.), and low-temperature (5° C.) conditions for 30 days. The presence or absence of deposits was then checked by visual inspection.

### Acid and Sugar Solution

### Acid and Sugar Solution Formulation

| | |
|---|---|
| 1. Sugar | 5.5 (%) |
| 2. Fructose glucose liquid sugar | 5.5 |
| 3. Citric acid (crystal) | 0.08 |
| 3. Trisodium citrate | pH adjustment (pH 3, 6 or 9) |

A total of 100% with water

As a comparative experiment, IQC and rutin (both in a simple form), and a powdery rutin composition (rutin:γ-CD=1:5, mole ratio) prepared according to the method of Example 1 were dissolved in the acid and sugar solution, and hot-packed in a 200-ml PET bottle (93° C.) as above. The drink so prepared was allowed to cool, and placed under 50° C., room temperature (25±2° C.), and low-temperature (5° C.) conditions for 30 days. The presence or absence of deposits was then observed.

The results are presented in Table 4. In the table, the symbol “+” means the presence of deposits, and “−” the absence of deposits. The symbol “×” means no dissolution in the acid and sugar solution.

TABLE 4

| | | | Flavonoid | | | | | Flavonoid | |
|---|---|---|---|---|---|---|---|---|---|
| | | | concentra- | | | | | concentra- | |
| | | Preservation | tion (%) | | | | Preservation | tion (%) | |
| Sample | pH | method | 0.10 | 0.05 | Sample | pH | method | 0.10 | 0.05 |
| Rutin | 3 | 50° C. | x | x | Rutin/γ-CD | 3 | 50° C. | + | + |
| | | Room temperature | x | x | | | Room temperature | + | + |
| | | Refrigerated | x | x | | | Refrigerated | + | + |
| | 6 | 50° C. | x | + | | 6 | 50° C. | + | − |
| | | Room temperature | x | + | | | Room temperature | + | + |
| | | Refrigerated | x | + | | | Refrigerated | + | + |
| | 9 | 50° C. | x | − | | 9 | 50° C. | − | − |
| | | Room temperature | + | + | | | Room temperature | − | − |
| | | Refrigerated | + | + | | | Refrigerated | − | − |
| IQC | 3 | 50° C. | x | x | IQC/γ-CD | 3 | 50° C. | − | − |
| | | Room temperature | x | x | | | Room temperature | − | − |
| | | Refrigerated | x | x | | | Refrigerated | − | − |
| | 6 | 50° C. | x | − | | 6 | 50° C. | − | − |
| | | Room temperature | x | + | | | Room temperature | − | − |
| | | Refrigerated | x | + | | | Refrigerated | − | − |
| | 9 | 50° C. | − | − | | 9 | 50° C. | − | − |
| | | Room temperature | + | − | | | Room temperature | − | − |
| | | Refrigerated | + | + | | | Refrigerated | − | − |

As the results show, the rutin as the inclusion product of γ-CD had slightly improved solubility in water, but was very unstable as demonstrated by the formation of deposits during preservation. In contrast, the IQC as the inclusion product of γ-CD had greatly improved solubility in water (see Experiment Example 1), and formed no deposits even when preserved under different pH and temperature conditions (pH of 3 to 9, low temperature to 50° C.), demonstrating that the solubility can be maintained over extended time periods, and, specifically, that the preservation solubility can be improved.

### Experiment Example 4

### Evaluation of Fading Suppressing Effect

The fading suppressing effects of the IQC and IQC composition were evaluated using various colorants presented in Table 5 below.

Specifically, a colorant-containing acidic drink of the formulation below was prepared. Then, the IQC of Preparation Example 1, and the IQC/γ-CD inclusion preparation prepared in Example 1 (IQC:γ-CD=1:5) were each added to the drink at a final IQC concentration of 0.01%. The drink was also adjusted by adding γ-CD (Wacker Chemical) at a final concentration of 0.14%.

### Formulation of Colorant-Containing Acidic Drink

| | |
|---|---|
| 1. Sugar | 5.5 (%) |
| 2. Fructose glucose liquid sugar | 5.5 |
| 3. Citric acid (anhydrous) | 0.08 |
| 4. Trisodium citrate | pH adjustment (pH 3.1) |
| 5. Color | Table 5 |

A total of 100% with water

TABLE 5

| Color | Product name | Amount added (%) |
|---|---|---|
| Red cabbage colorant | San Red** RCFU* | 0.05 |
| Cochineal colorant | San Red** NO. 1F* | 0.05 |
| Grape juice colorant | San Red** GRF* | 0.05 |
| Purple corn colorant | San Red** No. 5F* | 0.05 |
| Elderberry colorant | San Red** ELF* | 0.05 |
| Purple sweet potato | San Red** YMF* | 0.05 |
| colorant | | |
| β-carotene | Carotene Base NO. 35468* | 0.05 |
| Safflower yellow | San Yellow** NO. 2 SFU | 0.05 |
| colorant | | |
| Paprika colorant | Paprika Base NB* | 0.03 |

The drinks were irradiated with a fluorescent light (BIOTRON LH 300, NK System) or a UV Fade Meter (UV Long-Life Fade Meter FAL-3, Suga Test Instruments Co., Ltd.) for the durations presented in Table 6. Absorbance was measured before and after the irradiation, and the percentage remaining color (%) after the irradiation was calculated. Further, as a control test, a colorant-containing acidic drink was irradiated with the UV Fade Meter or fluorescent light as above without adding anything (no addition), and the percentage remaining color (%) after the irradiation was calculated.

TABLE 6

| | Fluorescence | Fade-Ometer |
|---|---|---|
| Color | irradiation time (D) | irradiation time (H) |
| Red cabbage colorant | 4 | 8 |
| Cochineal colorant | 6 | 6 |
| Grape juice colorant | 20 | 20 |
| Purple corn colorant | 1 | 4 |
| Elderberry colorant | 2 | 2 |
| Purple sweet potato colorant | 8 | 7 |
| β-carotene | 6 | 17 |
| Safflower yellow colorant | 4 | 4 |
| Paprika colorant | 1 | 3 |

The results for the fluorescent light irradiation, and the results for the UV Fade Meter irradiation are presented in Tables 7 and 8, respectively.

TABLE 7
Fluorescence irradiation

| Color | No addition | IQC | IQC-CD inclusion | γ-CD |
|---|---|---|---|---|
| Red cabbage colorant | 45.1 | 60.1 | 63.7 | 54.0 |
| Cochineal colorant | 65.7 | 75.6 | 86.9 | 71.5 |
| Grape juice colorant | 90.4 | 91.1 | 93.6 | 88.1 |
| Purple corn colorant | 69.4 | 76.4 | 83.9 | 69.2 |
| Elderberry colorant | 64.7 | 64.5 | 71.7 | 66.2 |
| Purple sweet potato | 68.2 | 76.9 | 79.9 | 75.6 |
| colorant | | | | |
| β-carotene | 53.4 | 71.2 | 82.8 | 60.6 |
| Safflower yellow | 65.3 | 72.8 | 86.0 | 69.2 |
| colorant | | | | |
| Paprika colorant | 75.5 | 91.3 | 95.0 | 90.3 |

TABLE 8
UV fade meter irradiation

| Color | No addition | IQC | IQC-CD inclusion | γ-CD |
|---|---|---|---|---|
| Red cabbage colorant | 24.2 | 50.8 | 67.6 | 25.1 |
| Cochineal colorant | 79.5 | 91.1 | 96.5 | 80.8 |
| Grape juice colorant | 88.6 | 93.1 | 97.6 | 88.7 |
| Purple corn colorant | 39.2 | 59.8 | 69.5 | 36.2 |
| Elderberry colorant | 63.6 | 70.2 | 85.5 | 65.2 |
| Purple sweet potato | 73.7 | 88.0 | 98.7 | 79.4 |
| colorant | | | | |
| β-carotene | 50.3 | 74.2 | 99.4 | 60.5 |
| Safflower yellow | 60.9 | 71.6 | 87.3 | 62.4 |
| colorant | | | | |
| Paprika colorant | 29.1 | 55.8 | 66.4 | 37.6 |

As can be seen from these results, it was confirmed that the IQC, capable of exhibiting the fading suppressing effect alone, can have an improved fading suppressing effect as an inclusion product of γ-CD.

### Experiment Example 5

### Evaluation of Flavor Deterioration Suppressing Effect

Various drinks mixed with the IQC or IQC composition were tested under severity conditions of light or heat to evaluate the flavor deterioration suppressing effect of the IQC or IQC composition.

### (1) Preparation of Drinks

Specifically, seven drinks were prepared in the formulations 1 to 7 below, and the IQC of Preparation Example 1, or the readily water-soluble IQC (sample 5) prepared in Experiment Example 1 was added to each drink in a final IQC concentration of 0.01% to obtain an IQC-rich drink. In the same manner, γ-CD (Wacker Chemical) was added to each drink in a final concentration of 0.14% to prepare a drink that contained γ-CD.

Formulation 1: Grape drink (pH 3.1)

| | |
|---|---|
| Fructose glucose liquid sugar | 10 (%) |
| Five-Times concentrated transparent | 4.4 |
| Concord grape juice | |
| Citric acid (anhydrous) | 0.15 |
| Trisodium citrate | 0.01 |
| Grape flavor NO. 63554* | 0.1 |

A total of 100% with water

Formulation 2: Lemon drink (pH 3.2)

| | |
|---|---|
| Sugar | 6 (%) |
| Citric acid (anhydrous) | 0.08 |
| Trisodium citrate | 0.08 |
| From-Concentrate lemon fruit juice | 3.0 |
| Lemon flavor NO. 2404* | 0.1 |

A total of 100% with water

Formulation 3: Grapefruit Drink (20% juice; pH 3.2)

| | |
|---|---|
| Grapefruit frozen juice 45 | 4 (%) |
| Fructose glucose liquid sugar | 5 |
| Sugar | 4 |
| Citric acid (anhydrous) | 0.1 |
| Trisodium citrate | 0.02 |
| L-ascorbic acid | 0.02 |
| Grapefruit flavor NO. 2512* | 0.1 |

A total of 100% with water

Formulation 4: Orange drink (20% juice %; pH 3.5)

| | |
|---|---|
| Citrus mixed fruit juice 53 | 4 (%) |
| Fructose glucose liquid sugar | 5 |
| Sugar | 4 |
| Citric acid (anhydrous) | 0.1 |
| Trisodium citrate | 0.02 |
| L-ascorbic acid | 0.02 |
| Orange flavor NO. 2410* | 0.1 |

A total of 100% with water

Formulation 5: Coffee drink (pH 6.3)

| | |
|---|---|
| Sugar | 6.5 (%) |
| Coffee extract | 55 |
| Whole powdered milk | 0.76 |
| Powdered skim milk | 1.11 |
| Emulsifier (Homogen** No. 352*) | 0.05 |
| Sodium bicarbonate | 0.07 |
| Coffee flavor NO. 63378* | 0.05 |

A total of 100% with water

Formulation 6: Milk tea (pH 6.8)

| | |
|---|---|
| Black tea leaf | 0.4 (%) |
| Milk | 6 |
| Whole powdered milk | 1.4 |
| Sugar | 6 |
| Emulsifier (Homogen** | 0.167 |
| NO. 1890*) | |

A total of 100% with water

Formulation 7: Sour milk beverage (pH 3.3)

| | |
|---|---|
| Fermented milk | 10 (%) |
| Sugar | 7 |
| Citric acid (anhydrous) | 0.07 |
| Soybean polysaccharide (SM-1200*) | 0.2 |
| Pectin (SM-600(A)*) | 0.07 |
| Emulsifier (Homogen** NO. 2429*) | 0.1 |
| Yogurt flavor NO. 61127* | 0.1 |
| ST flavor NO. 9702* | 0.03 |

A total of 100% with water

### (2) Evaluation Test for Flavor Deterioration Suppression

The drinks prepared as above were tested under the severity conditions presented in Table 9, and changes in drink flavor after the test were examined by six panelists in a sensory evaluation. Evaluations were made according to the following criteria on the scale of 1 (stress tested drink (blank) without any addition) to 5 (each drink before the test).

Evaluation criteria
5: No change from before the test
4: Slight change from before the test
3: Little change from before the test
2: Notable change from before the test
1: Considerable change from before the test (comparable to the blank)

Table 9 presents means values of the scored points evaluated by the six panelists.

TABLE 9

| | Severity condition | IQC | IQC-CD | γ-CD |
|---|---|---|---|---|
| Formulation 1: | Fluorescent light (20000 1x, | 2.7 | 3.2 | 2.6 |
| | 21 hr, 15° C.) | | | |
| Grape drink | Heat (60° C., 64 hr) | 2.2 | 2.4 | 2.1 |
| Formulation 2: | Fluorescent light (20000 1x, | 2.6 | 3.1 | 2.4 |
| | 21 hr, 10° C.) | | | |
| Lemon drink | Heat (60° C., 64 hr) | 2.2 | 2.6 | 2.5 |
| Formulation 3: | Fluorescent light (20000 1x, | 3.1 | 3.5 | 2.9 |
| | 21 hr, 15° C.) | | | |
| Grapefruit | Heat (60° C., 64 hr) | 2.5 | 2.7 | 2.5 |
| drink | | | | |
| Formulation 4: | Fluorescent light (20000 1x, | 2.9 | 3.1 | 2.7 |
| | 18 hr, 15° C.) | | | |
| Orange drink | Heat (60° C., 64 hr) | 2.7 | 3.0 | 2.4 |
| Formulation 5: | Fluorescent light (20000 1x, | 2.9 | 3.5 | 2.9 |
| Coffee | 18 hr, 15° C.) | | | |
| Formulation 6: | Fluorescent light (20000 1x, | 2.9 | 3.1 | 2.7 |
| Milk tea | 18 hr, 15° C.) | | | |
| Formulation 7: | Fluorescent light (20000 1x, | 2.9 | 3.3 | 2.4 |
| Sour milk | 18 hr, 15° C.) | | | |
| beverage | | | | |

These results confirmed that the use of IQC as an inclusion product of γ-CD, rather than by itself, can further suppress flavor deterioration in the all drinks. Specifically, it was found that the IQC composition of the present invention has a better flavor deterioration suppressing effect than the IQC itself. IQC deposits were observed within several hours from the preparation of the drink in Samples in which the IQC was added as it is.

### Experiment Example 6

### Absorption in the Body

A powdery IQC composition (sample 5) was prepared according to the method of Example 1, using IQC and γ-CD at a ratio of 1:5 (mole ratio).

A person (n=1) fasted with no breakfast was orally administered once with IQC (375 mg) or the powdery IQC composition (equivalent of 375 mg IQC) prepared above. After 1, 2, and 3 hours from the administration, bloods were collected using heparin as anti-coagulant from the person, and centrifuged to obtain plasmas.

About 29,000 unit/ml β-glucuronidase (20 μl) dissolved in 0.58 M sodium acetate buffer (pH 4.9) was added to the plasma (180 μl), and the mixture was left unattended at 37° C. for 30 min. After adding 1 M AsA/10 mM sodium metaphosphate (50 μl, in terms of phosphoric acid) and 20 μg/ml diosmetin (9 μl), the mixture was frozen at −80° C., and freeze dried overnight. The freeze dried sample was suspended in MeOH (1 ml), and centrifuged at 15,000 rpm for 10 min. The supernatant was concentrated and dried, and dissolved in MeOH (90 μl). After adding 200 mM HCl (90 μl), the solution was centrifuged at 15,000 rpm for 10 min to collect the supernatant. The IQC metabolites quercetin, isorhamnetin, and tamarixetin in the plasma were then quantified by HPLC under the conditions below. From the total amount, the administered IQC (open square) and IQC composition (solid square) were compared to each other with regard to the metabolic absorption.
HPLC conditions
HPLC: Agilent
Column: YMC Pack ODS-AQ (φ4.6×250 mm)
Mobile phase: 0.1% phosphoric acid/MeCN MeCN 25%→35% (0-10 min), 35% (10-24 min), 100% (24-29 min), 25% (29-35 min)
Flow rate: 1.0 ml/min
Temperature: 30° C.
Detection: UV 370 nm
Injected amount: 100 μl

The results are presented in Table 10 and FIG. 3.

TABLE 10

| | | | IQC |
|---|---|---|---|
| Flavonoid in | | | inclusion |
| plasma | Blood collection time (h) | IQC (375 mg) | (IQC 375 mg) |
| Quercetin | 0 | 0.02 | 0.06 |
| | 1 | 0.01 | 0.50 |
| | 2 | 0.15 | 0.64 |
| | 3 | 0.15 | 0.46 |
| Isorhamnetin | 0 | 0 | 0 |
| | 1 | 0 | 0.01 |
| | 2 | 0.02 | 0.03 |
| | 3 | 0.01 | 0.06 |
| Tamarixetin | 0 | 0 | 0.02 |
| | 1 | 0 | 0.08 |
| | 2 | 0.06 | 0.15 |
| | 3 | 0.03 | 0.13 |
| Total | 0 | 0.02 | 0.08 |
| | 1 | 0.01 | 0.59 |
| | 2 | 0.22 | 0.83 |
| | 3 | 0.20 | 0.65 |

(μg/ml)

As can be seen from the results, the IQC (non-inclusion) had low absorption in the body, and the concentration in the blood plasma reached 0.2 μg/ml only after 2 hours from the administration. In contrast, the IQC as an inclusion product of γ-CD had improved absorption, and was quickly absorbed immediately after the administration. At hour 2 post administration, the amount of the absorbed isoquercitrin was 4 times greater in the administration of the IQC inclusion than that in the administration of the IQC alone. Further, while the IQC had an AUC of 0.34 (μg/ml)·hr, the inclusion product with γ-CD had an AUC of 1.785 (μg/ml)·hr.

1. A readily water-soluble isoquercitrin composition that comprises γ-cyclodextrin in a proportion of 2 to 10 moles per mole of isoquercitrin.

2. A readily water-soluble isoquercitrin composition according to claim 1, wherein the isoquercitrin is an inclusion product of the γ-cyclodextrin.

3. A readily water-soluble isoquercitrin composition according to claim 2, wherein the readily water-soluble isoquercitrin composition is a color deterioration suppressing agent.

4. A readily water-soluble isoquercitrin composition according to claim 2, wherein the readily water-soluble isoquercitrin composition is a flavor deterioration suppressing agent.

5. A colorant preparation that comprises a colorant with a readily water-soluble isoquercitrin composition of claim 3.

6. A scented product that comprises a flavor component with a readily water-soluble isoquercitrin composition of claim 4.

7. A liquid or semiliquid edible composition that comprises a readily water-soluble isoquercitrin composition of claim 2 in the state of being dissolved in water or in aqueous ethanol.

8. An edible composition according to claim 7, wherein the edible composition is a drink.

9. An edible composition obtained by solidifying a liquid or semiliquid edible composition of claim 7.

10. A method for producing a readily water-soluble isoquercitrin composition of claim 2, the method comprising including the isoquercitrin in γ-cyclodextrin in a proportion of 2 to 10 moles of the γ-cyclodextrin per mole of the isoquercitrin.

11. A method according to claim 10, comprising subjecting the mixture that contains the γ-cyclodextrin and the isoquercitrin in a proportion of 2 to 10 moles of the γ-cyclodextrin per mole of the isoquercitrin to the following steps A or B,
A. (1) dissolving the mixture in a heated aqueous solution, and (2) drying the aqueous solution,
B. (1) dissolving the mixture in a heated aqueous solution, and clarifying the aqueous solution, and (2) drying the aqueous solution.

12. A method for improving the solubility of isoquercitrin in water and for suppressing the deposition of isoquercitrin in an aqueous solution of pH 3 to 9,
the method comprising including isoquercitrin in γ-cyclodextrin in a proportion of 2 to 10 moles of the γ-cyclodextrin per mole of the isoquercitrin.

13. (canceled)

14. (canceled)

15. A method for improving body's absorption of isoquercitrin taken orally, the method comprising including isoquercitrin in γ-cyclodextrin in a proportion of 2 to 10 moles of the γ-cyclodextrin per mole of the isoquercitrin.

16. A readily water-soluble isoquercitrin composition according to claim 1, wherein the readily water-soluble isoquercitrin composition is a color deterioration suppressing agent.

17. A readily water-soluble isoquercitrin composition according to claim 1, wherein the readily water-soluble isoquercitrin composition is a flavor deterioration suppressing agent.

18. A colorant preparation that comprises a colorant with a readily water-soluble isoquercitrin composition of claim 16.

19. A scented product that comprises a flavor component with a readily water-soluble isoquercitrin composition of claim 17.

20. A liquid or semiliquid edible composition that comprises a readily water-soluble isoquercitrin composition of claim 1 in the state of being dissolved in water or in aqueous ethanol.

21. An edible composition according to claim 20, wherein the edible composition is a drink.

22. An edible composition obtained by solidifying a liquid or semiliquid edible composition of claim 20.

23. A method for producing a readily water-soluble isoquercitrin composition of claim 1, the method comprising including the isoquercitrin in γ-cyclodextrin in a proportion of 2 to 10 moles of the γ-cyclodextrin per mole of the isoquercitrin.

24. A method according to claim 23, comprising subjecting the mixture that contains the γ-cyclodextrin and the isoquercitrin in a proportion of 2 to 10 moles of the γ-cyclodextrin per mole of the isoquercitrin to the following steps A or B,
A. (1) dissolving the mixture in a heated aqueous solution, and (2) drying the aqueous solution,
B. (1) dissolving the mixture in a heated aqueous solution, and clarifying the aqueous solution, and (2) drying the aqueous solution.
